(19) United States
(12) Patent Application Publication
(10) Pub. No.: US 2025/0032180 A1
(43) Pub. Date: Jan. 30, 2025

(54) SYSTEMS AND METHODS THAT FACILITATE TRANSDUCER SELECTION FOR ACTIVATION BASED ON TISSUE CONTACT INFORMATION

(71) Applicant: KARDIUM INC., Burnaby (CA)

(72) Inventors: Douglas Wayne GOERTZEN, New Westminster (CA); Daniel Robert WEINKAM, Coquitlam (CA); Kevin Marc CHAPLIN, Vancouver (CA)

(21) Appl. No.: 18/905,309

(22) Filed: Oct. 3, 2024

Related U.S. Application Data
(63) Continuation of application No. PCT/CA2023/050572, filed on Apr. 27, 2023.
(60) Provisional application No. 63/336,123, filed on Apr. 28, 2022.

(51) Int. Cl. A61B 18/14; A61B 18/00; A61B 18/08; A61B 90/00

(52) U.S. Cl. CPC A61B 18/1492; A61B 18/082; A61B 2018/0016; A61B 2018/00267; A61B 2018/00577; A61B 2018/00654; A61B 2090/065

(57) ABSTRACT

Tissue contact information indicating degrees of tissue contact between various transducers of a transducer-based device and a tissue surface may be received. A group of at least some of such transducers may be machine-selected as having each transducer in the group meeting or exceeding a threshold degree of tissue contact as indicated by an analysis of the tissue contact information. User input may be received that indicates one or more transducers to be added to or removed from the machine-selected group of transducers, resulting in a modified selected set of one or more transducers. The modified selected set of one or more transducers may then be activated.

### CROSS-REFERENCE TO RELATED APPLICATIONS

This application is a Bypass Continuation Application of International Application No. PCT/CA2023/050572, filed Apr. 27, 2023, which claims the benefit of U.S. Provisional Application No. 63/336, 123, filed Apr. 28, 2022, the entire disclosure of each of the applications cited in this sentence is hereby incorporated herein by reference.

### TECHNICAL FIELD

Aspects of this disclosure generally are related to systems and methods for selecting, activating, or selecting and activating transducers, such systems and methods applicable to, among other things, medical systems.

### BACKGROUND

Cardiac surgery was initially undertaken using highly invasive open procedures. A sternotomy, which is a type of incision in the center of the chest that separates the sternum was typically employed to allow access to the heart. In the past several decades, more and more cardiac operations are performed using intravascular or percutaneous techniques, where access to inner organs or other tissue is gained via a catheter.

Intravascular or percutaneous surgeries benefit patients by reducing surgery risk, complications, and recovery time. However, the use of intravascular or percutaneous technologies also raises some particular challenges. Medical devices used in intravascular or percutaneous surgery need to be deployed via catheter systems which significantly increase the complexity of the device structure. As well, doctors do not have direct visual contact with the medical devices once the devices are positioned within the body.

One example of where intravascular or percutaneous medical techniques have been employed is in the treatment of a heart disorder called atrial fibrillation. Atrial fibrillation is a disorder in which spurious electrical signals cause an irregular heartbeat. Atrial fibrillation has been treated with various methods including a technique known as “PV (pulmonary vein) isolation”. Research has shown that atrial fibrillation typically begins in the pulmonary veins or at the point where they attach to the left atrium. There are typically four major pulmonary veins, and some or all may be a focal point for activity that may cause atrial fibrillation. During this procedure, physicians create specific patterns of lesions in the left or right atrium to block various paths taken by the spurious electrical signals. The patterns of lesions may include a pattern of one or more lesions that encircle at least one of the pulmonary veins. Lesions were originally created using incisions, but are now typically created by ablating the tissue with various techniques including pulsed field ablation (PFA) (also known as irreversible electroporation), radiofrequency (RF) ablation, microwave ablation, laser ablation, and cryogenic ablation. Lesion formation may be performed with a high success rate under the direct vision that is provided in open procedures, but is relatively complex to perform intravascularly or percutaneously because of the difficulty in creating the lesions in the correct locations. Various problems, potentially leading to severe adverse events, may occur if the lesions are placed incorrectly. It is particularly important to know the position of the various transducers which will be creating the lesions relative to cardiac features such as the pulmonary veins and mitral valve. The continuity, transmurality and placement of the lesion patterns that are formed can impact the ability to block paths taken within the heart by spurious electrical signals. Further, it is typically desired to not “over-ablate” or to needlessly apply ablative energy to tissue regions that are not required to be ablated to provide effective therapy. For example, stiff left atrial syndrome is a possible complication that may occur several years after catheter ablation due to scarring with reduction or loss of left atrial compliance and contractility. This complication may occur after extensive ablation within the left atrium. Stiff left atrial syndrome may be defined as the combination of heart failure symptoms and left atrial dysfunction in the absence of a significant mitral valve pathology.

Another commonly important factor for effective therapy, particularly in the context of major operations such as operations performed on the heart, is duration of the procedure. All else being equal, it is typically preferable to reduce procedure time, as longer procedures tend to increase risk to the patient.

In this regard, the present inventors recognized that there is a need for improved intra-bodily-cavity transducer-based device systems or control mechanisms thereof with improved transducer selection capabilities to help achieve appropriate transducer activations, such as appropriate lesion formation, reducing the risk of over ablating or needlessly ablating tissue, and to help reduce procedure times.

### SUMMARY

At least the above-discussed need is addressed, and technical solutions are achieved by various embodiments of the present invention. According to some embodiments, a system may be summarized as including an input-output device system, a memory device system storing a program, and a data processing device system communicatively connected to the input-output device system and the memory device system. According to some embodiments, the data processing device system may be configured at least by the program at least to cause, via the input-output device system, reception of tissue contact information indicating degrees of tissue contact between various transducers of a plurality of transducers of a transducer-based device and a tissue surface. In some embodiments, the data processing device system may be configured at least by the program at least to cause, based at least on an analysis of the tissue contact information, a machine selection of a machine-selected group of transducers of the plurality of transducers. According to some embodiments, the machine selection may select the machine-selected group of transducers as having each transducer in the machine-selected group of transducers being associated with an indication of contact with the tissue surface based at least on the analysis of the tissue contact information and as having each transducer in the machine-selected group of transducers meeting or exceeding a threshold degree of tissue contact as indicated by the analysis of the tissue contact information. In some embodiments, the data processing device system may be configured at least by the program at least to cause, via the input-output device system and after the machine selection, reception of user input indicating a user-deselected transducer set. According to various embodiments, each transducer in the user-deselected transducer set may be a transducer in the machine-selected group of transducers that is to be deselected. According to various embodiments, the machine-selected group of transducers excluding the user-deselected transducer set may form a modified selected transducer set. In some embodiments, the data processing device system may be configured at least by the program at least to cause, via the input-output device system and at least after the machine selection and after the reception of the user input indicating the user-deselected transducer set, activation of the modified selected transducer set.

In some embodiments, the machine-selected group of transducers may consist of some, but not all, of the transducers of the plurality of transducers. In some embodiments, each transducer of the plurality of transducers meeting or exceeding the threshold degree of tissue contact as indicated by the analysis of the tissue contact information may be selected by the machine selection for inclusion in the machine-selected group of transducers. In some embodiments, the activation of the modified selected transducer set may include concurrent activation of at least two transducers in the modified selected transducer set. In some embodiments, the activation of the modified selected transducer set may include a transmission of energy between at least two transducers in the modified selected transducer set. In some embodiments, the energy may be configured to cause tissue ablation. In some embodiments, the tissue ablation is pulsed field ablation. In some embodiments, the tissue ablation is thermal ablation.

In some embodiments, the activation of the modified selected transducer set may include a transmission of energy from each of at least one transducer in the modified selected transducer set, the energy configured to cause tissue ablation. In some embodiments, the tissue ablation is pulsed field ablation. In some embodiments, the tissue ablation is thermal ablation.

In some embodiments, the activation of the modified selected transducer set may include causing a sensing of electrophysiological information by each of at least one transducer in the modified selected transducer set.

In some embodiments, the plurality of transducers of the transducer-based device may be arranged in a spatial distribution. In some embodiments, at least one transducer of the plurality of transducers is located between the user-deselected transducer set and the modified selected transducer set in the spatial distribution. In some embodiments, the at least one transducer is not included in the user-deselected transducer set and is not included in the modified selected transducer set.

In some embodiments, the plurality of transducers of the transducer-based device may be arranged in a spatial distribution. In some embodiments, each particular transducer in the modified selected transducer set is located in the spatial distribution adjacently to another transducer in the modified selected transducer set, and at least a first transducer in the user-deselected transducer set is not located in the spatial distribution adjacently to any transducer in the modified selected transducer set. In some embodiments, at least a second transducer in the user-deselected transducer set may be located in the spatial distribution adjacently to at least one transducer in the modified selected transducer set. In some embodiments, each transducer in the user-deselected transducer set is not located in the spatial distribution adjacently to any transducer in the modified selected transducer set. In some embodiments, the transducers in the modified selected transducer set may be arranged in a ring-shaped arrangement in the spatial distribution. In some embodiments the transducers in the modified selected transducer set may surround, in the spatial distribution, a particular transducer set of the plurality of transducers. According to various embodiments, each transducer in the particular transducer set is not included in the modified selected transducer set. In some embodiments, each transducer in the particular transducer set is not included in the user-deselected transducer set. In some embodiments, the transducers in the modified selected transducer set may be arranged in a clustered arrangement in the spatial distribution.

In some embodiments, the plurality of transducers of the transducer-based device may be arranged in a spatial distribution. In some embodiments, the user-deselected transducer set may be a user-deselected group of transducers. In some embodiments, the transducers in the modified selected transducer set may be arranged in a first particular arrangement in the spatial distribution. In some embodiments, the transducers in the user-deselected group of transducers may be arranged in a second particular arrangement in the spatial distribution, the second particular arrangement separated from the first particular arrangement in the spatial distribution. In some embodiments, the transducers in the user-deselected group of transducers may be arranged in a second particular arrangement in the spatial distribution, the second particular arrangement protruding from the first particular arrangement in the spatial distribution.

In some embodiments, the number of transducers in the user-deselected transducer set may be lower than the number of transducers in the modified selected transducer set. In some embodiments, the user-deselected transducer set may be a user-deselected group of transducers.

In some embodiments, the machine-selected group of transducers may include a first transducer and a second transducer, the first transducer having a first degree of tissue contact as indicated by the analysis of the tissue contact information and the second transducer having a second degree of tissue contact as indicated by the analysis of the tissue contact information. In some embodiments, the second degree of tissue contact may be greater than the first degree of tissue contact. In some embodiments, the user-deselected transducer set may include the first transducer. In some embodiments, the user-deselected transducer set may exclude the second transducer.

In some embodiments, the machine-selected group of transducers may include a first transducer and a second transducer, the first transducer having a first degree of tissue contact as indicated by the analysis of the tissue contact information and the second transducer having a second degree of tissue contact as indicated by the analysis of the tissue contact information. In some embodiments, the second degree of tissue contact may be equal to or greater than the first degree of tissue contact, and in some embodiments, the user-deselected transducer set may include the second transducer.

Various systems may include combinations and subsets of all the systems summarized above or otherwise described herein.

According to some embodiments, a system may be summarized as including an input-output device system, a memory device system storing a program, and a data processing device system communicatively connected to the input-output device system and the memory device system. According to some embodiments, the data processing device system may be configured at least by the program at least to cause, via the input-output device system, reception of tissue contact information indicating degrees of tissue contact between various transducers of a plurality of transducers of a transducer-based device and a tissue surface. In some embodiments, the data processing device system may be configured at least by the program at least to cause, based at least on an analysis of the tissue contact information, a machine selection of a machine-selected group of transducers of the plurality of transducers. In some embodiments, the machine selection may select the machine-selected group of transducers as having each transducer in the group of transducers being associated with an indication of contact with the tissue surface based at least on the analysis of the tissue contact information and as having each transducer in the machine-selected group of transducers meeting or exceeding a threshold degree of tissue contact as indicated by the analysis of the tissue contact information. In some embodiments, the data processing device system may be configured at least by the program at least to cause, via the input-output device system and after the machine selection, reception of user input indicating a user selection of a user-selected transducer set. In some embodiments, each transducer in the user-selected transducer set may not be any transducer in the machine-selected group of transducers, and each transducer in the user-selected transducer set may have less than the threshold degree of tissue contact. In some embodiments, the data processing device system may be configured at least by the program at least to cause, via the input-output device system, activation of the machine-selected group of transducers and the user-selected transducer set. According to various embodiments, the activation may be initiated in a state in which both the machine-selected group of transducers and the user-selected transducer set are in a selected state.

In some embodiments, the machine-selected group of transducers and the user-selected transducer set, in combination, may consist of some, but not all, of the transducers of the plurality of transducers. In some embodiments, each transducer of the plurality of transducers meeting or exceeding the threshold degree of tissue contact as indicated by the analysis of the tissue contact information may be selected by the machine selection for inclusion in the machine-selected group of transducers. In some embodiments, the activation of the machine-selected group of transducers and the user-selected transducer set may be caused at least in response to both the machine selection and the user selection. In some embodiments, the data processing device system may be configured at least by the program at least to record in the memory device system a first indication indicating that the machine-selected group of transducers is in the selected state, in response to the machine selection. In some embodiments, the data processing device system may be configured at least by the program at least to record in the memory device system a second indication indicating that the user-selected transducer set is in the selected state, in response to the user selection. In some embodiments, the activation of the machine-selected group of transducers and the user-selected transducer set may be initiated in a particular state in which the first indication and the second indication concurrently exist as recorded in the memory device system.

In some embodiments, the activation of the machine-selected group of transducers and the user-selected transducer set may include concurrent activation of (a) at least one transducer in the machine-selected group of transducers and (b) at least one transducer in the user-selected transducer set. In some embodiments, the activation of the machine-selected group of transducers and the user-selected transducer set may include a transmission of energy between (a) at least one transducer in the machine-selected group of transducers, and (b) at least one transducer in the user-selected transducer set. In some embodiments the energy may be sufficient to cause tissue ablation. In some embodiments, the tissue ablation may be pulsed field ablation. In some embodiments, the tissue ablation may be thermal ablation.

In some embodiments, the machine-selected group of transducers and the user-selected transducer set form a combined group of transducers, and the activation of the machine-selected group of transducers and the user-selected transducer set may include causing a sensing of electrophysiological information by each of at least one transducer in the combined group of transducers. In some embodiments, the at least one transducer in the combined group of transducers may include at least a first transducer in the machine-selected group of transducers and at least a second transducer in the user-selected transducer set.

In some embodiments, the plurality of transducers of the transducer-based device may be arranged in a spatial distribution. In some embodiments, the transducers in the machine-selected group of transducers may be arranged in a ring-shaped arrangement in the spatial distribution. In some embodiments, the transducers in the machine-selected group of transducers and the user-selected transducer set may be arranged in a ring-shaped arrangement in the spatial distribution. In some embodiments, the transducers in the machine-selected group of transducers and the user-selected transducer set may surround a particular transducer set of the plurality of transducers in the spatial distribution. According to some embodiments, each transducer in the particular transducer set may not be included in either the machine-selected group of transducers or the user-selected transducer set. In some embodiments, the transducers in the machine-selected group of transducers and the user-selected transducer set may be arranged in a clustered arrangement in the spatial distribution.

In some embodiments, the number of transducers in the user-selected transducer set may be lower than the number of transducers in the machine-selected group of transducers. In some embodiments, the user-selected transducer set may be a user-selected group of transducers.

Various systems may include combinations and subsets of all the systems summarized above or otherwise described herein.

Various embodiments of the present invention may include systems, devices, or machines that are or include combinations or subsets of any one or more of the systems, devices, or machines and associated features thereof summarized above or otherwise described herein (which should be deemed to include the figures).

Further, all or part of any one or more of the systems, devices, or machines summarized above or otherwise described herein or combinations or sub-combinations thereof may implement or execute all or part of any one or more of the processes or methods described herein or combinations or sub-combinations thereof.

According to some embodiments, a method may be executed by a data processing device system according to a program stored by a communicatively connected memory device system, the data processing device system also communicatively connected to an input-output device system, and the method may include: receiving, via the input-output device system, tissue contact information indicating degrees of tissue contact between various transducers of a plurality of transducers of a transducer-based device and a tissue surface; machine-selecting, based at least on an analysis of the tissue contact information, a machine-selected group of transducers of the plurality of transducers, the machine selection selecting the machine-selected group of transducers as having each transducer in the machine-selected group of transducers being associated with an indication of contact with the tissue surface based at least on the analysis of the tissue contact information and as having each transducer in the machine-selected group of transducers meeting or exceeding a threshold degree of tissue contact as indicated by the analysis of the tissue contact information; receiving, via the input-output device system and after the machine selection, user input indicating a user-deselected transducer set, each transducer in the user-deselected transducer set being a transducer in the machine-selected group of transducers that is to be deselected, the machine-selected group of transducers excluding the user-deselected transducer set forming a modified selected transducer set; and activating, via the input-output device system and at least after the machine selection and after the reception of the user input indicating the user-deselected transducer set, the modified selected transducer set.

According to some embodiments, a method may be executed by a data processing device system according to a program stored by a communicatively connected memory device system, the data processing device system also communicatively connected to an input-output device system, and the method may include: receiving, via the input-output device system, tissue contact information indicating degrees of tissue contact between various transducers of a plurality of transducers of a transducer-based device and a tissue surface; machine selecting, based at least on an analysis of the tissue contact information, a machine-selected group of transducers of the plurality of transducers, the machine selection selecting the machine-selected group of transducers as having each transducer in the group of transducers being associated with an indication of contact with the tissue surface based at least on the analysis of the tissue contact information and as having each transducer in the machine-selected group of transducers meeting or exceeding a threshold degree of tissue contact as indicated by the analysis of the tissue contact information; receiving, via the input-output device system and after the machine selection, user input indicating a user selection of a user-selected transducer set, each transducer in the user-selected transducer set not being any transducer in the machine-selected group of transducers, and each transducer in the user-selected transducer set having less than the threshold degree of tissue contact; and activating, via the input-output device system, the machine-selected group of transducers and the user-selected transducer set, the activation initiated in a state in which both the machine-selected group of transducers and the user-selected transducer set are in a selected state.

It should be noted that various embodiments of the present invention include variations of the methods or processes summarized above or otherwise described herein (which should be deemed to include the figures) and, accordingly, are not limited to the actions described or shown in the figures or their ordering, and not all actions shown or described are required according to various embodiments. According to various embodiments, such methods may include more or fewer actions and different orderings of actions. Any of the features of all or part of any one or more of the methods or processes summarized above or otherwise described herein may be combined with any of the other features of all or part of any one or more of the methods or processes summarized above or otherwise described herein.

In addition, a computer program product may be provided that includes program code portions for performing some or all of any one or more of the methods or processes and associated features thereof described herein, when the computer program product is executed by a computer or other computing device or device system. Such a computer program product may be stored on one or more computer-readable storage mediums, also referred to as one or more computer-readable data storage mediums or a computer-readable storage medium system.

According to some embodiments, one or more computer-readable storage mediums may store a program executable by a data processing device system communicatively connected to an input-output device system, and the program may include first reception instructions configured to cause, via the input-output device system, reception of tissue contact information indicating degrees of tissue contact between various transducers of a plurality of transducers of a transducer-based device and a tissue surface. The program may include machine selection instructions configured to cause, based at least on an analysis of the tissue contact information, a machine selection of a machine-selected group of transducers of the plurality of transducers, the machine selection selecting the machine-selected group of transducers as having each transducer in the machine-selected group of transducers being associated with an indication of contact with the tissue surface based at least on the analysis of the tissue contact information and as having each transducer in the machine-selected group of transducers meeting or exceeding a threshold degree of tissue contact as indicated by the analysis of the tissue contact information. The program may include second reception instructions configured to cause, via the input-output device system and after the machine selection, reception of user input indicating a user-deselected transducer set, each transducer in the user-deselected transducer set being a transducer in the machine-selected group of transducers that is to be deselected, the machine-selected group of transducers excluding the user-deselected transducer set forming a modified selected transducer set. The program may include activation instructions configured to cause, via the input-output device system and at least after the machine selection and after the reception of the user input indicating the user-deselected transducer set, activation of the modified selected transducer set.

According to some embodiments, one or more computer-readable storage mediums may store a program executable by a data processing device system communicatively connected to an input-output device system, and the program may include first reception instructions configured to cause, via the input-output device system, reception of tissue contact information indicating degrees of tissue contact between various transducers of a plurality of transducers of a transducer-based device and a tissue surface. The program may include machine selection instructions configured to cause, based at least on an analysis of the tissue contact information, a machine selection of a machine-selected group of transducers of the plurality of transducers, the machine selection selecting the machine-selected group of transducers as having each transducer in the group of transducers being associated with an indication of contact with the tissue surface based at least on the analysis of the tissue contact information and as having each transducer in the machine-selected group of transducers meeting or exceeding a threshold degree of tissue contact as indicated by the analysis of the tissue contact information. The program may include second reception instructions configured to cause, via the input-output device system and after the machine selection, reception of user input indicating a user selection of a user-selected transducer set, each transducer in the user-selected transducer set not being any transducer in the machine-selected group of transducers, and each transducer in the user-selected transducer set having less than the threshold degree of tissue contact. The program may include activation instructions configured to cause, via the input-output device system, activation of the machine-selected group of transducers and the user-selected transducer set, the activation initiated in a state in which both the machine-selected group of transducers and the user-selected transducer set are in a selected state.

In some embodiments, each of any of one or more or all of the computer-readable data storage mediums or medium systems (also referred to as processor-accessible memory device systems) described herein is a non-transitory computer-readable (or processor-accessible) data storage medium or medium system (or memory device system) including or consisting of one or more non-transitory computer-readable (or processor-accessible) storage mediums (or memory devices) storing the respective program(s) which may configure a data processing device system to execute some or all of any of one or more of the methods or processes described herein.

Further, any of all or part of one or more of the methods or processes and associated features thereof discussed herein may be implemented or executed on or by all or part of a device system, apparatus, or machine, such as all or a part of any of one or more of the systems, apparatuses, or machines described herein or a combination or sub-combination thereof.

### BRIEF DESCRIPTION OF THE DRAWINGS

It is to be understood that the attached drawings are for purposes of illustrating aspects of various embodiments and may include elements that are not to scale.

FIG. 1 includes a schematic representation of a transducer-activation system according to various example embodiments, the transducer-activation system including a data processing device system, an input-output device system, and a memory device system.

FIG. 2 includes a cutaway diagram of a heart showing a transducer-based device percutaneously placed in a left atrium of the heart, according to various example embodiments.

FIG. 3A includes a partially schematic representation of a medical system according to various example embodiments, the medical system including a data processing device system, an input-output device system, a memory device system, and a transducer-based device including a plurality of transducers and an expandable structure shown in a delivery or unexpanded configuration.

FIG. 3B includes a portion of the medical system of FIG. 3A as viewed from a different viewing direction.

FIG. 3C includes the representation of the medical system of FIGS. 3A and 3B with the expandable structure shown in a deployed or expanded configuration, according to some embodiments.

FIG. 3D includes a portion of the medical system of FIG. 3C as viewed from a different viewing direction, according to some embodiments.

FIG. 4 includes a schematic representation of a transducer-based device that includes a flexible circuit structure, according to various example embodiments.

FIG. 5A includes a graphical interface providing a graphical representation, according to various example embodiments, a depiction of at least a portion of a transducer-based device including a plurality of transducer graphical elements depicted among the graphical representation.

FIG. 5B includes the graphical interface of FIG. 5A with the portion of the transducer-based device depicted as viewed from a different viewing direction than that shown in FIG. 5A, according to some embodiments.

FIG. 5C includes the graphical representation provided by the graphical interface of FIG. 5A with the addition of various between graphical elements positioned between various ones of the transducer graphical elements, according to some embodiments.

FIG. 5D includes the graphical representation provided by the graphical interface of FIG. 5C but with the portion of the transducer-based device depicted as viewed from a different viewing direction than that shown in FIG. 5C, according to some embodiments.

FIG. 5E includes the graphical representation provided by the graphical interface of FIGS. 5C and 5D depicted with one particular form of two-dimensional representation in accordance with various example embodiments.

FIG. 5F includes the graphical representation provided by the graphical interface of FIGS. 5A and 5B depicted with another particular form of two-dimensional representation in accordance with various example embodiments.

FIG. 5G includes a graphical interface providing a graphical representation, according to various example embodiments, including a depiction of at least a portion of a transducer-based device including a plurality of transducer graphical elements depicted among the graphical representation, and the depiction of the at least the portion of the transducer-based device within an envelope representing at least a portion of a bodily cavity, according to some embodiments.

FIG. 5H illustrates a graphical representation of at least a portion of a transducer-based device, the graphical representation of the at least the portion of the transducer-based device including transducer graphical elements corresponding to transducers of the transducer-based device, and the transducer graphical elements including a visual characteristic set or a lack of at least a portion thereof indicating various states of tissue contact or a state of no-tissue contact experienced by the corresponding transducers of the transducer-based device, according to some embodiments.

FIG. 5I illustrates the graphical representation of FIG. 5H in a state in which some of the transducer graphical elements include a visual characteristic set indicating a machine-selection of the corresponding transducers of the transducer-based device, the machine-selection selecting each of such transducers as meeting or exceeding a threshold degree of tissue contact, according to some embodiments.

FIG. 5J illustrates the graphical representation of FIG. 5I in a state after which user input has been received, the user input indicating a user-deselection of some of the transducers that were machine-selected in the state of FIG. 5I, according to some embodiments.

FIG. 5K illustrates the graphical representation of FIG. 5J in a state in which additional user input has been received, the additional user input indicating a user-selection of some of the transducers that were not machine-selected in the state of FIG. 5I, according to some embodiments.

FIGS. 6A and 6B include block diagrams of various methods for activating transducers of a transducer-based device, according to some embodiments.

### DETAILED DESCRIPTION

The above-discussed need in the art are addressed and technical solutions are achieved according to various embodiments of the present invention. In various embodiments, efficient transducer selection mechanisms are provided, such mechanisms facilitating a quicker selection of appropriate transducers, compared to conventional techniques, to perform efficient and effective transducer activations and to reduce procedure duration, among other benefits. In the case where the transducer activations perform tissue ablation and lesion formation, risk of over ablating or needlessly ablating tissue is reduced. In the case where the transducer activations perform a sensing function or mapping function, transducers that are most suitable to perform such functions may be efficiently selected, thereby reducing procedure time. In some embodiments, tissue contact information is received by a data processing device system, the tissue contact information indicating degrees of tissue contact between various transducers of a plurality of transducers of a transducer-based device and a tissue surface. In some embodiments, based at least on an analysis of the tissue contact information, a machine selection of a machine-selected group of transducers of the plurality of transducers occurs, the machine selection selecting the machine-selected group of transducers as having each transducer in the group of transducers having at least a threshold degree of tissue contact as indicated by the analysis of the tissue contact information, the threshold degree of tissue contact indicating contact with the tissue surface. By having a machine or automatic selection of transducers that meet or exceed a threshold degree of tissue contact, the user may be quickly presented with a group of transducers that are likely to be deemed to be able to effectively activate, such as to form a tissue lesion through ablation with sufficient contact with the tissue or to sense a tissue characteristic with sufficient contact with the tissue. From there, the user may then add transducers to or subtract transducers from the machine-selected group of transducers, resulting in a final transducer set that the user desires to activate. Accordingly, a transducer-based device or control system thereof, according to various embodiments of the present invention, provides efficient mechanisms to quickly achieve a final transducer set to perform effective and efficient activation, such as tissue ablation activation or tissue sensing activation, thereby reducing procedure risk (such as avoiding over ablating tissue, needlessly ablating tissue, or avoiding poor sensing results) and reducing overall procedure time. These and other features and benefits of various embodiments of the present invention will become apparent after reading the remainder of this disclosure and reviewing the figures.

In the following description, certain specific details are set forth in order to provide a thorough understanding of various embodiments of the invention. However, one skilled in the art will understand that the invention may be practiced at a more general level without these details. In other instances, well-known structures have not been shown or described in detail to avoid unnecessarily obscuring descriptions of various embodiments of the invention.

Any reference throughout this specification to “one embodiment”, “an embodiment”, “an example embodiment”, “an illustrated embodiment”, “a particular embodiment”, and the like means that a particular feature, structure or characteristic described in connection with the embodiment is included in at least one embodiment. Thus, any appearance of the phrase “in one embodiment”, “in an embodiment”, “in an example embodiment”, “in this illustrated embodiment”, “in this particular embodiment”, or the like in this specification is not necessarily always referring to one embodiment or a same embodiment. Furthermore, the particular features, structures or characteristics of different embodiments may be combined in any suitable manner to form one or more other embodiments.

Unless otherwise explicitly noted or required by context, the word “or” is used in this disclosure in a non-exclusive sense. In addition, unless otherwise explicitly noted or required by context, the word “set” is intended to mean one or more. For example, the phrase, “a set of objects” means one or more of the objects. In some embodiments, the word “subset” is intended to mean a set having the same or fewer elements of those present in the subset's parent or superset. In other embodiments, the word “subset” is intended to mean a set having fewer elements of those present in the subset's parent or superset. In this regard, when the word “subset” is used, some embodiments of the present invention utilize the meaning that “subset” has the same or fewer elements of those present in the subset's parent or superset, and other embodiments of the present invention utilize the meaning that “subset” has fewer elements of those present in the subset's parent or superset.

Further, the phrase “at least” is or may be used herein at times merely to emphasize the possibility that other elements may exist besides those explicitly listed. However, unless otherwise explicitly noted (such as by the use of the term “only”) or required by context, non-usage herein of the phrase “at least” nonetheless includes the possibility that other elements may exist besides those explicitly listed. For example, the phrase, ‘based at least on A’ includes A as well as the possibility of one or more other additional elements besides A. In the same manner, the phrase, ‘based on A’ includes A, as well as the possibility of one or more other additional elements besides A. However, the phrase, ‘based only on A’ includes only A. Similarly, the phrase ‘configured at least to A’ includes a configuration to perform A, as well as the possibility of one or more other additional actions besides A. In the same manner, the phrase ‘configured to A’ includes a configuration to perform A, as well as the possibility of one or more other additional actions besides A. However, the phrase, ‘configured only to A’ means a configuration to perform only A.

The word “device”, the word “machine”, the word “system”, and the phrase “device system” all are intended to include one or more physical devices or sub-devices (e.g., pieces of equipment) that interact to perform one or more functions, regardless of whether such devices or sub-devices are located within a same housing or different housings. However, it may be explicitly specified according to various embodiments that a device or machine or device system resides entirely within a same housing to exclude embodiments where the respective device, machine, system, or device system resides across different housings. The word “device” may equivalently be referred to as a “device system” in some embodiments, and the word “system” may equivalently be referred to as a “device system” in some embodiments.

Further, the phrase “in response to” may be used in this disclosure. For example, this phrase may be used in the following context, where an event A occurs in response to the occurrence of an event B. In this regard, such phrase includes, for example, that at least the occurrence of the event B causes or triggers the event A.

The phrase “thermal ablation” as used in this disclosure refers, in some embodiments, to an ablation method in which destruction of tissue occurs by hyperthermia (elevated tissue temperatures) or hypothermia (depressed tissue temperatures). Thermal ablation may include radiofrequency (“RF”) ablation, microwave ablation, or cryo-ablation by way of non-limiting example. RF ablation energy waveforms can take various forms. For example, in some RF ablation embodiments, energy is provided in the form of a continuous waveform. In some RF ablation embodiments, energy is provided in the form of discrete energy applications (e.g., in the form of a duty cycled waveform).

The phrase “pulsed field ablation” (“PFA”) as used in this disclosure refers, in some embodiments, to an ablation method that employs high voltage pulse delivery in a unipolar or bipolar fashion in proximity to target tissue. In some embodiments, each high voltage pulse may be referred to as a discrete energy application. In some embodiments, a grouped plurality of high voltages pulses may be referred to as a discrete energy application. Each high voltage pulse can be a monophasic pulse including a single polarity, or a biphasic pulse including a first component having a first particular polarity and a second component having a second particular polarity opposite the first particular polarity. In some embodiments, the second component of the biphasic pulse follows immediately after the first component of the biphasic pulse. In some embodiments, the first and second components of the biphasic pulse are temporally separated by a relatively small time interval. In some embodiments, each high voltage pulse may include a multiphasic pulse, such as a triphasic pulse, that includes a first component having a first particular polarity, a second component having a second particular polarity opposite the first particular polarity, and a third component having a third particular polarity that is the same as the first particular polarity. The electric field applied by the high voltage pulses in PFA physiologically changes the tissue cells to which the energy is applied (e.g., puncturing or perforating the cell membrane to form various pores therein). If a lower field strength is established, the formed pores may close in time and cause the cells to maintain viability (e.g., a process sometimes referred to as reversible electroporation). If the field strength that is established is greater, then permanent, and sometimes larger, pores form in the tissue cells, the pores allowing loss of control of ion concentration gradients (e.g., both inwards and outwards) thereby resulting in cell death (e.g., a process sometimes referred to as irreversible electroporation).

According to some embodiments, the word “fluid” as used in this disclosure should be understood to include any fluid that can be contained within a bodily cavity or can flow into or out of, or both into and out of a bodily cavity via one or more bodily openings positioned in fluid communication with the bodily cavity. In the case of cardiac applications, fluid such as blood will flow into and out of various intracardiac cavities (e.g., a left atrium or a right atrium).

According to some embodiments, the words “bodily opening” as used in this disclosure should be understood to include a naturally occurring bodily opening or channel or lumen; a bodily opening or channel or lumen formed by an instrument or tool using techniques that can include, but are not limited to, mechanical, thermal, electrical, chemical, and exposure or illumination techniques; a bodily opening or channel or lumen formed by trauma to a body; or various combinations of one or more of the above. Various elements having respective openings, lumens, or channels and positioned within the bodily opening (e.g., a catheter sheath) may be present in various embodiments. These elements may provide a passageway through a bodily opening for various devices employed in various embodiments.

The words “bodily cavity” as used in this disclosure should be understood to mean a cavity in a body, in some embodiments. The bodily cavity may be a cavity or chamber provided in a bodily organ (e.g., an intracardiac cavity of a heart).

The word “tissue” as used in some embodiments in this disclosure should be understood to include any surface-forming tissue that is used to form a surface of a body or a surface within a bodily cavity, a surface of an anatomical feature or a surface of a feature associated with a bodily opening positioned in fluid communication with the bodily cavity.

The tissue can include part, or all, of a tissue wall or membrane that defines a surface of the bodily cavity. In this regard, the tissue can form an interior surface of the cavity that surrounds a fluid within the cavity. In the case of cardiac applications, tissue can include tissue used to form an interior surface of an intracardiac cavity such as a left atrium or a right atrium.

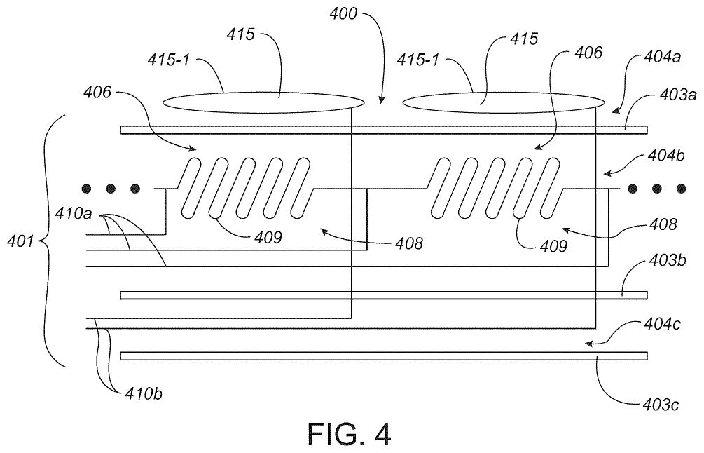

According to some embodiment, the term “transducer” as used in this disclosure should be interpreted broadly as any device capable of transmitting or delivering energy, distinguishing between fluid and tissue, sensing temperature, creating heat, ablating tissue, sensing, sampling or measuring electrical activity of a tissue surface (e.g., sensing, sampling or measuring intracardiac electrograms, or sensing, sampling or measuring intracardiac voltage data), stimulating tissue, or any combination thereof. A transducer may convert input energy of one form into output energy of another form. Without limitation, a transducer may include an electrode that functions as, or as part of, a sensing device included in the transducer, an energy delivery device included in the transducer, or both a sensing device and an energy delivery device included in the transducer. A transducer may be constructed from several parts, which may be discrete components or may be integrally formed. In this regard, although transducers, electrodes, or both transducers and electrodes are referenced with respect to various embodiments, it is understood that other transducers or transducer elements may be employed in other embodiments. It is understood that a reference to a particular transducer in various embodiments may also imply a reference to an electrode, as an electrode may be part of the transducer as shown, e.g., at least with FIG. 4 discussed below.

The term “activation” as used in this disclosure, according to some embodiments, should be interpreted broadly as making active a particular function as related to various transducers disclosed in this disclosure. Particular functions may include, but are not limited to, tissue ablation (e.g., PFA), sensing, sampling or measuring electrophysiological activity (e.g., sensing, sampling or measuring intracardiac electrogram information or sensing, sampling or measuring intracardiac voltage data), sensing, sampling or measuring temperature and sensing, sampling or measuring electrical characteristics (e.g., tissue impedance or tissue conductivity). For example, in some embodiments, activation of a tissue ablation function of a particular transducer is initiated by causing energy sufficient for tissue ablation from an energy source device system to be delivered to the particular transducer. Also, in this example, the activation can last for a duration of time concluding when the ablation function is no longer active, such as when energy sufficient for the tissue ablation is no longer provided to the particular transducer. In some contexts, however, the word “activation” can merely refer to the initiation of the activating of a particular function, as opposed to referring to both the initiation of the activating of the particular function and the subsequent duration in which the particular function is active. In these contexts, the phrase or a phrase similar to “activation initiation” may be used.

In the following description, some embodiments of the present invention may be implemented at least in part by a data processing device system or a controller system configured by a software program. Such a program may equivalently be implemented as multiple programs, and some, or all, of such software program(s) may be equivalently constructed in hardware. In this regard, reference to “a program” should be interpreted to include one or more programs.

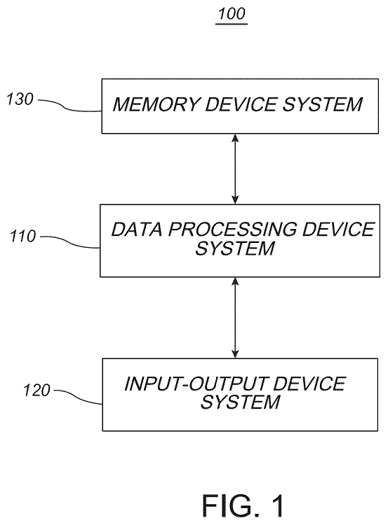

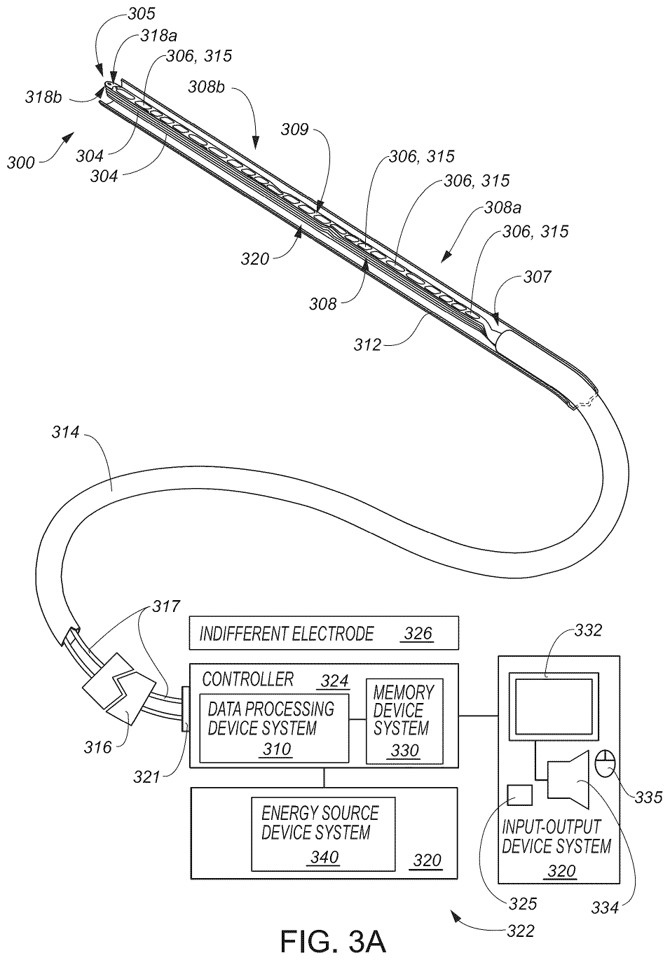

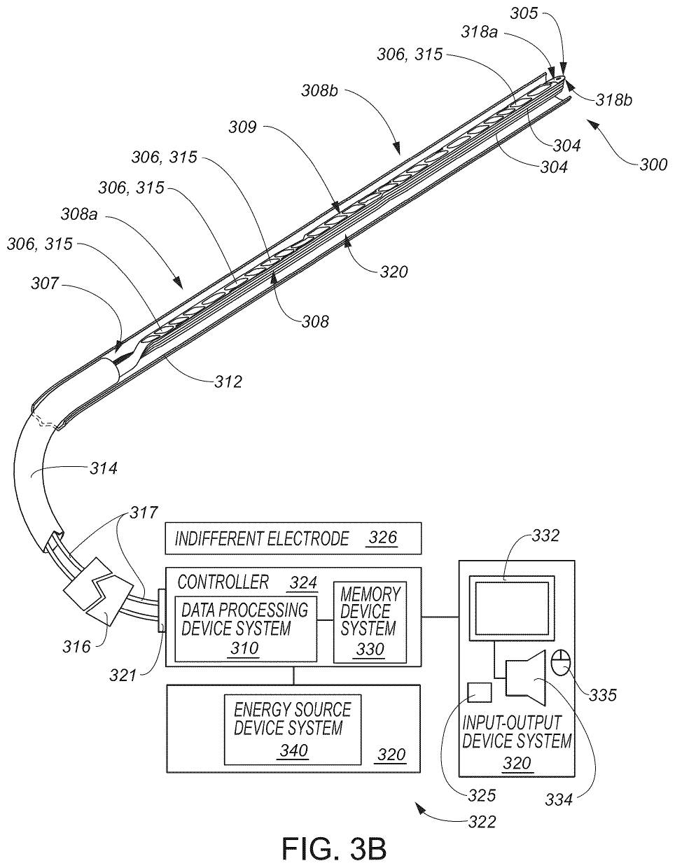

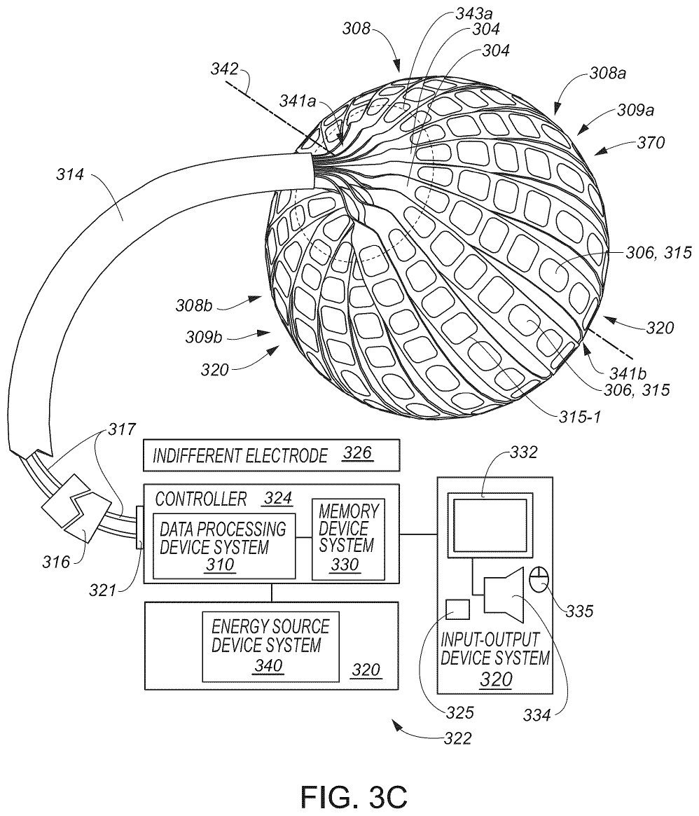

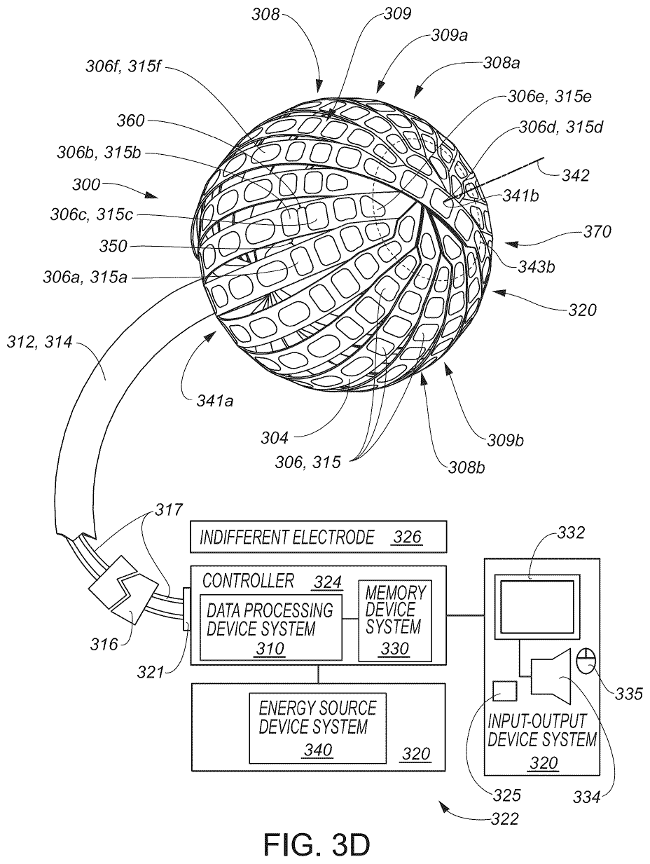

According to some embodiments, the term “program” in this disclosure should be interpreted to include one or more programs including a set of instructions or modules that can be executed by one or more components in a system, such as a controller system or a data processing device system, in order to cause the system to perform one or more operations. The set of instructions or modules may be stored by any kind of memory device, such as those described subsequently with respect to the memory device system 130 or 330 shown in at least FIGS. 1 and 3, respectively. In addition, this disclosure may describe or similarly describe that the instructions or modules of a program are configured to cause the performance of an action. The phrase “configured to” in this context is intended to include, for example, at least (a) instructions or modules that are presently in a form executable by one or more data processing devices to cause performance of the action (e.g., in the case where the instructions or modules are in a compiled and unencrypted form ready for execution), and (b) instructions or modules that are presently in a form not executable by the one or more data processing devices, but could be translated into the form executable by the one or more data processing devices to cause performance of the action (e.g., in the case where the instructions or modules are encrypted in a non-executable manner, but through performance of a decryption process, would be translated into a form ready for execution). Such descriptions should be deemed to be equivalent to describing that the instructions or modules are configured to cause the performance of the action. The word “module” may be defined as a set of instructions. In some instances, this disclosure describes that the instructions or modules of a program perform a function. The word “program” and the word “module” may each be interpreted to include multiple sub-programs or multiple sub-modules, respectively. In this regard, reference to a program or a module may be considered to refer to multiple programs or multiple modules.

Further, it is understood that information or data may be operated upon, manipulated, or converted into different forms as it moves through various devices or workflows. In this regard, unless otherwise explicitly noted or required by context, it is intended that any reference herein to information or data or the like includes modifications to that information or data. For example, “data X” may be encrypted for transmission, and a reference to “data X” is intended to include both its encrypted and unencrypted forms, unless otherwise required or indicated by context. For another example, “image information Y” may undergo a noise filtering process, and a reference to “image information Y” is intended to include both the pre-processed form and the noise-filtered form, unless otherwise required or indicated by context. In other words, both the pre-processed form and the noise-filtered form are considered to be “image information Y”, unless otherwise required or indicated by context. In order to stress this point, the phrase “or a derivative thereof” or the like may be used herein. Continuing the preceding example, the phrase “image information Y or a derivative thereof” refers to both the pre-processed form and the noise-filtered form of “image information Y”, unless otherwise required or indicated by context, with the noise-filtered form potentially being considered a derivative of “image information Y”. However, non-usage of the phrase “or a derivative thereof” or the like nonetheless includes derivatives or modifications of information or data unless otherwise explicitly noted or required by context.

In some embodiments, the phrase “graphical representation” used herein is intended to include a visual representation presented via a display device system and may include computer-generated text, graphics, animations, or one or more combinations thereof, which may include one or more visual representations originally generated, at least in part, by an image-capture device, such as computerized tomography (“CT”) scan images, magnetic resonance imaging (“MRI”) images, or images created from a navigation system (e.g., electric potential navigation system or an electromagnetic navigation system), according to some embodiments. The graphical representation may include various entities depicted in a two-dimensional manner. The graphical representation may include various entities depicted in a three-dimensional manner, in some embodiments.

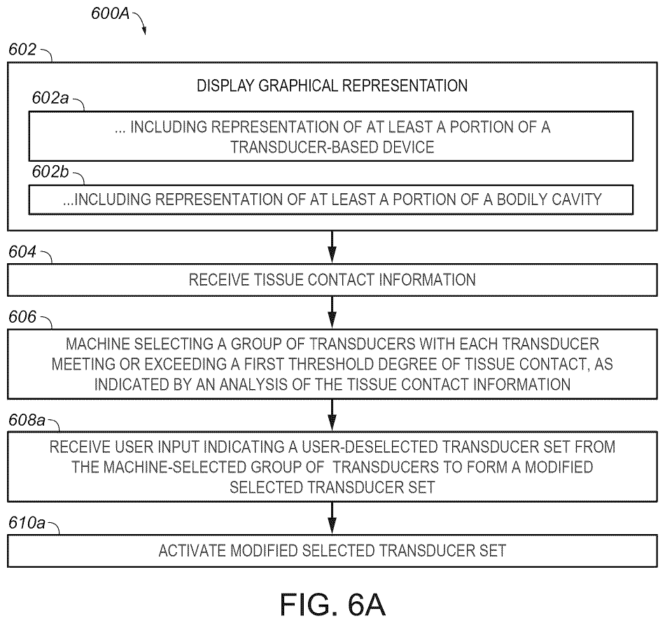

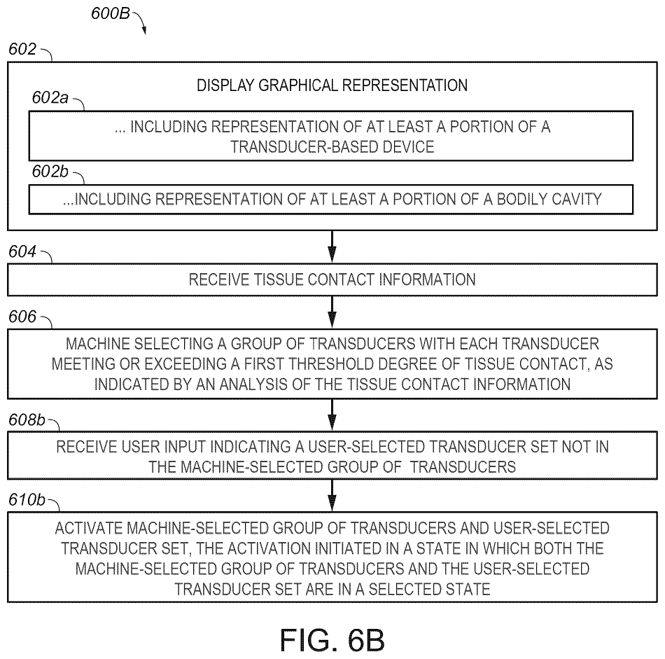

Example methods are described herein with respect to FIGS. 6A and 6B. Such figures include blocks associated with actions, computer-executable instructions, or both, according to various embodiments. It should be noted that the respective instructions associated with any such blocks therein need not be separate instructions and may be combined with other instructions to form a combined instruction set. The same set of instructions may be associated with more than one block. In this regard, the block arrangement shown in each of the method figures herein is not limited to an actual structure of any program or set of instructions or required ordering of method tasks, and such method figures, according to some embodiments, merely illustrate the tasks that instructions are configured to perform, for example, upon execution by a data processing device system in conjunction with interactions with one or more other devices or device systems.

Each of the phrases “derived from” or “derivation of” or “derivation thereof” or the like may be used herein, according to some embodiments, to mean to come from at least some part of a source, be created from at least some part of a source, or be developed as a result of a process in which at least some part of a source forms an input. For example, a data set derived from some particular portion of data may include at least some part of the particular portion of data, or may be created from at least part of the particular portion of data, or may be developed in response to a data manipulation process in which at least part of the particular portion of data forms an input. In some embodiments, a data set may be derived from a subset of the particular portion of data. In some embodiments, the particular portion of data is analyzed to identify a particular subset of the particular portion of data, and a data set is derived from the subset. In various ones of these embodiments, the subset may include some, but not all, of the particular portion of data. In some embodiments, changes in least one part of a particular portion of data may result in changes in a data set derived at least in part from the particular portion of data.

In this regard, each of the phrases “derived from” or “derivation of” or “derivation thereof” or the like may be used herein merely to emphasize the possibility that such data or information may be modified or subject to one or more operations. For example, if a device generates first data for display, the process of converting the generated first data into a format capable of being displayed may alter the first data. This altered form of the first data may be considered a derivative or derivation of the first data. For instance, the first data may be a one-dimensional array of numbers, but the display of the first data may be a color-coded bar chart representing the numbers in the array. For another example, if the above-mentioned first data is transmitted over a network, the process of converting the first data into a format acceptable for network transmission or understanding by a receiving device may alter the first data. As before, this altered form of the first data may be considered a derivative or derivation of the first data. For yet another example, generated first data may undergo a mathematical operation, a scaling, or a combining with other data to generate other data that may be considered derived from the first data. In this regard, it can be seen that data is commonly changing in form or being combined with other data throughout its movement through one or more data processing device systems, and any reference to information or data herein is intended in some embodiments to include these and like changes, regardless of whether or not the phrase “derived from” or “derivation of” or “derivation thereof” or the like is used in reference to the information or data. As indicated above, usage of the phrase “derived from” or “derivation of” or “derivation thereof” or the like merely emphasizes the possibility of such changes. Accordingly, in some embodiments, the addition of or deletion of the phrase “derived from” or “derivation of” or “derivation thereof” or the like should have no impact on the interpretation of the respective data or information. For example, the above-discussed color-coded bar chart may be considered a derivative of the respective first data or may be considered the respective first data itself.

In some embodiments, the term “adjacent”, the term “proximate”, and the like refer at least to a sufficient closeness between the objects or events defined as adjacent, proximate, or the like, to allow the objects or events to interact in a designated way. For example, in the case of physical objects, if object A performs an action on an adjacent or proximate object B, objects A and B would have at least a sufficient closeness to allow object A to perform the action on object B. In this regard, some actions may require contact between the associated objects, such that if object A performs such an action on an adjacent or proximate object B, objects A and B would be in contact, for example, in some instances or embodiments where object A needs to be in contact with object B to successfully perform the action. In some embodiments, the term “adjacent”, the term “proximate”, and the like additionally or alternatively refer to objects or events that do not have another substantially similar object or event between them. For example, object or event A and object or event B could be considered adjacent or proximate (e.g., physically or temporally) if they are immediately next to each other (with no other object or event between them) or are not immediately next to each other but no other object or event that is substantially similar to object or event A, object or event B, or both objects or events A and B, depending on the embodiment, is between them. In the context of graphical elements, discussed at various points in this description, two graphical elements may be considered adjacent, in some embodiments, in which the two graphical elements have no other graphical element between them that is the same in form to any of the two graphical elements. In the context of graphical elements, discussed at various points in this description, two graphical elements may be considered adjacent, in some embodiments, in which the two graphical elements have no other graphical element between them that is the same in function to any of the two graphical elements. In the context of transducer graphical elements, discussed at various points in this description, two transducer graphical elements may be considered adjacent, in some embodiments, in which the two transducer graphical elements have no other transducer graphical element between them. In some embodiments, the term “adjacent”, the term “proximate”, and the like additionally or alternatively refer to at least a sufficient closeness between the objects or events defined as adjacent, proximate, and the like, the sufficient closeness being within a range that does not place any one or more of the objects or events into a different or dissimilar region or time period, or does not change an intended function of any one or more of the objects or events or of an encompassing object or event that includes a set of the objects or events. Different embodiments of the present invention adopt different ones or combinations of the above definitions. Of course, however, the term “adjacent”, the term “proximate”, and the like are not limited to any of the above example definitions, according to some embodiments. In addition, the term “adjacent” and the term “proximate” do not have the same definition, according to some embodiments.

FIG. 1 schematically illustrates a portion of a transducer-activation system or controller system thereof 100 that may be employed to at least select, control, activate, or monitor a function or activation of a number of electrodes or transducers (e.g., ablation transducers configured to cause thermal ablation or ablation transducers configured to cause PFA), according to some embodiments. The system 100 includes a data processing device system 110, an input-output device system 120, and a processor-accessible memory device system 130. The processor-accessible memory device system 130 and the input-output device system 120 are communicatively connected to the data processing device system 110. According to some embodiments, various components such as data processing device system 110, input-output device system 120, and processor-accessible memory device system 130 form at least part of a controller system (e.g., controller system 324 shown in FIG. 3).

The data processing device system 110 includes one or more data processing devices that implement or execute, in conjunction with other devices, such as those in the system 100, various methods and functions described herein, including those described with respect to methods exemplified in FIGS. 6A and 6B. Each of the phrases “data processing device”, “data processor”, “processor”, “controller”, “computing device”, “computer” and the like is intended to include any data or information processing device, such as a central processing unit (CPU), a control circuit, a desktop computer, a laptop computer, a mainframe computer, a tablet computer, a personal digital assistant, a cellular or smart phone, and any other device for processing data, managing data, or handling data, whether implemented with electrical, magnetic, optical, quantum, or biological components, or otherwise.

The memory device system 130 includes one or more processor-accessible memory devices configured to store one or more programs and information, including the program(s) and information needed to execute the methods or functions described herein, including those described with respect to FIGS. 6A and 6B. The memory device system 130 may be a distributed processor-accessible memory device system including multiple processor-accessible memory devices communicatively connected to the data processing device system 110 via a plurality of computers and/or devices. However, the memory device system 130 need not be a distributed processor-accessible memory system and, consequently, may include one or more processor-accessible memory devices located within a single data processing device or housing.

Each of the phrases “processor-accessible memory” and “processor-accessible memory device” and the like is intended to include any processor-accessible data storage device or medium, whether volatile or nonvolatile, electronic, magnetic, optical, or otherwise, including but not limited to, registers, hard disk drives, Compact Discs, DVDs, flash memories, ROMs, and RAMs. In some embodiments, each of the phrases “processor-accessible memory” and “processor-accessible memory device” is intended to include or be a processor-accessible (or computer-readable) data storage medium. In some embodiments, each of the phrases “processor-accessible memory” and “processor-accessible memory device” may include or may be a non-transitory processor-accessible (or computer-readable) data storage medium. In some embodiments, the processor-accessible memory device system 130 may include or may be a non-transitory processor-accessible (or computer-readable) data storage medium system. In some embodiments, the memory device system 130 may include or may be a non-transitory processor-accessible (or computer-readable) storage medium system or data storage medium system including or consisting of one or more non-transitory processor-accessible (or computer-readable) storage or data storage mediums.

The phrase “communicatively connected” is intended to include any type of connection, whether wired or wireless, between devices, data processors, or programs between which data may be communicated. Further, the phrase “communicatively connected” is intended to include a connection between devices or programs within a single data processor or computer, a connection between devices or programs located in different data processors or computers, and a connection between devices not located in data processors or computers at all. In this regard, although the memory device system 130 is shown separately from the data processing device system 110 and the input-output device system 120, one skilled in the art will appreciate that the memory device system 130 may be located completely or partially within the data processing device system 110 or the input-output device system 120. Further in this regard, although the input-output device system 120 is shown separately from the data processing device system 110 and the memory device system 130, one skilled in the art will appreciate that such system may be located completely or partially within the data processing system 110 or the memory device system 130, for example, depending upon the contents of the input-output device system 120. Further still, the data processing device system 110, the input-output device system 120, and the memory device system 130 may be located entirely within the same device or housing or may be separately located, but communicatively connected, among different devices or housings. In the case where the data processing device system 110, the input-output device system 120, and the memory device system 130 are located within the same device, the system 100 of FIG. 1 can be implemented by a single application-specific integrated circuit (ASIC) in some embodiments.

The input-output device system 120 may include a mouse, a keyboard, a touch screen, another computer, or any device or combination of devices from which a desired selection, desired information, instructions, or any other data is input to the data processing device system 110. The input-output device system 120 may include a user-activatable control system that is responsive to a user action. The user-activatable control system may include at least one control element that may be activated or deactivated on the basis of a particular user action. The input-output device system 120 may include any suitable interface for receiving information, instructions or any data from other devices and systems described in various ones of the embodiments. In this regard, the input-output device system 120 may include various ones of other systems described in various embodiments. For example, the input-output device system 120 may include at least a portion of a transducer-based device. The phrase “transducer-based device” or “transducer-based device system” is intended to include one or more physical systems that include various transducers. A PFA device system that includes one or more transducers may be considered a transducer-based device or device system, according to some embodiments.

The input-output device system 120 also may include an image generating device system, a display device system, a speaker or audio output device system, a computer, a processor-accessible memory device system, a network-interface card or network-interface circuitry, or any device or combination of devices to which information, instructions, or any other data is output by the data processing device system 110. In this regard, the input-output device system 120 may include various other devices or systems described in various embodiments. The input-output device system 120 may include any suitable interface for outputting information, instructions, or data to other devices and systems described in various ones of the embodiments. If the input-output device system 120 includes a processor-accessible memory device, such memory device may, or may not, form part, or all, of the memory device system 130. The input-output device system 120 may include any suitable interface for outputting information, instructions, or data to other devices and systems described in various ones of the embodiments. In this regard, the input-output device system 120 may include various other devices or systems described in various embodiments.

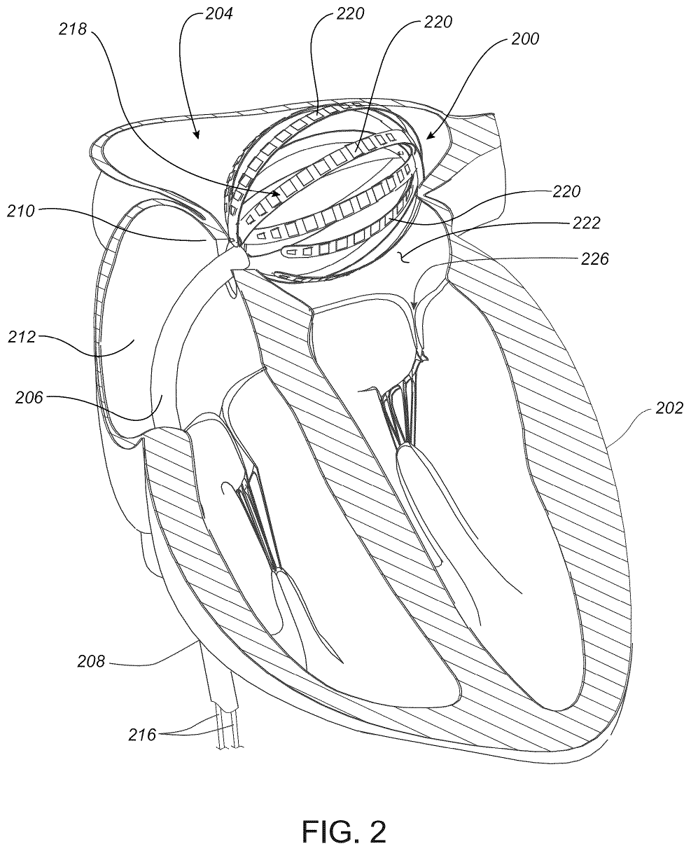

Various embodiments of transducer-based devices are described herein in this disclosure. Some of the described devices are PFA devices that are percutaneously or intravascularly deployed. Some of the described devices are movable between a delivery or unexpanded configuration (e.g., FIGS. 3A and 3B discussed below) in which a portion of the device is sized for passage through a bodily opening leading to a bodily cavity, and an expanded or deployed configuration (e.g., FIGS. 2, 3C, and 3D discussed below) in which the portion of the device has a size too large for passage through the bodily opening leading to the bodily cavity. An example of an expanded or deployed configuration, in some embodiments, is when the portion of the transducer-based device is in its intended-deployed-operational state, which may be inside the bodily cavity when, e.g., performing an intended therapeutic or diagnostic procedure for a patient, or which may be outside the bodily cavity when, e.g., performing testing, quality control, or other evaluation of the device. Another example of the expanded or deployed configuration, in some embodiments, is when the portion of the transducer-based device is being changed from the delivery configuration to the intended-deployed-operational state to a point where the portion of the device now has a size too large for passage through the bodily opening leading to the bodily cavity.

In some example embodiments, the device includes transducers that sense characteristics (e.g., convective cooling, permittivity, force) that distinguish between fluid, such as a fluidic tissue (e.g., blood), and tissue forming an interior surface of the bodily cavity. Such sensed characteristics can allow a medical system to map the cavity, for example, using positions of openings or ports into and out of the cavity to determine a position or orientation (e.g., pose), or both of the portion of the device in the bodily cavity. In some example embodiments, the described systems employ a navigation system or electro-anatomical mapping system including electromagnetic-based systems and electropotential-based systems to determine a positioning of a portion of a device in a bodily cavity. In some example embodiments, the described devices are part of a transducer-activation system capable of ablating tissue in a desired pattern within the bodily cavity using various techniques (e.g., via thermal ablation, PFA, etc., according to various embodiments).

In some example embodiments, the devices are capable of sensing various cardiac functions (e.g., electrophysiological activity including intracardiac voltages). In some example embodiments, the devices are capable of providing stimulation (e.g., electrical stimulation) to tissue within the bodily cavity. Electrical stimulation may include pacing.

FIG. 2 is a representation of a transducer-based device 200 useful in investigating or treating a bodily organ, for example, a heart 202, according to at least one example embodiment.

Transducer-based device 200 can be percutaneously or intravascularly inserted into a portion of the heart 202, such as an intra-cardiac cavity like left atrium 204. In this example, the transducer-based device 200 is part of a catheter 206 inserted via the inferior vena cava 208 and penetrating through a bodily opening in transatrial septum 210 from right atrium 212. (In this regard, transducer-based devices or device systems described herein that include a catheter may also be referred to as catheter devices or catheter-based devices, in some embodiments). In other embodiments, other paths may be taken.

Catheter 206 includes an elongated flexible rod or shaft member appropriately sized to be delivered percutaneously or intravascularly. Various portions of catheter 206 may be steerable. Catheter 206 may include one or more lumens. The lumen(s) may carry one or more communications or power paths, or both. For example, the lumens(s) may carry one or more electrical conductors 216 (two shown). Electrical conductors 216 provide electrical connections to transducer-based device 200 that are accessible externally from a patient in which the transducer-based device 200 is inserted.

Transducer-based device 200 includes a frame or structure 218 which assumes an unexpanded configuration for delivery to left atrium 204. Structure 218 is expanded (e.g., shown in a deployed or expanded configuration in FIG. 2) upon delivery to left atrium 204 to position a plurality of transducers 220 (three called out in FIG. 2) proximate the interior surface formed by tissue 222 of left atrium 204. In some embodiments, at least some of the transducers 220 are used to sense a physical characteristic of a fluid (e.g., blood) or tissue 222, or both, that may be used to determine a position or orientation (e.g., pose), or both, of a portion of a device 200 within, or with respect to left atrium 204. For example, transducers 220 may be used to determine a location of pulmonary vein ostia or a mitral valve 226, or both. In some embodiments, at least some of the transducers 220 may be used to selectively ablate portions of the tissue 222. For example, some of the transducers 220 may be used to ablate a pattern around the bodily openings, ports or pulmonary vein ostia, for instance to reduce or eliminate the occurrence of atrial fibrillation. In some embodiments, at least some of the transducers 220 are used to ablate cardiac tissue. In some embodiments, at least some of the transducers 220 are used to sense or sample intra-cardiac voltage data or sense or sample intra-cardiac electrogram data. In some embodiments, at least some of the transducers 220 are used to sense or sample intra-cardiac voltage data or sense or sample intra-cardiac electrogram data while at least some of the transducers 220 are concurrently ablating cardiac tissue. In some embodiments, at least one of the sensing or sampling transducers 220 is provided by at least one of the ablating transducers 220. In some embodiments, at least a first one of the transducers 220 senses or samples intra-cardiac voltage data or intra-cardiac electrogram data at a location at least proximate to a tissue location ablated by at least a second one of the transducers 220. In some embodiments, the first one of the transducers 220 is other than the second one of the transducers 220.

FIGS. 3A, 3B, 3C, and 3D (collectively, FIG. 3) include a transducer-based device system (e.g., a portion thereof shown schematically) that includes a transducer-based device 300 according to some embodiments. Transducer-based device 300 includes a plurality of elongate members 304 (not all of the elongate members called out in each of FIGS. 3A, 3B, 3C and 3D) and a plurality of transducers 306 (not all of the transducers called out in FIG. 3) (some of the transducers 306 called out in FIG. 3D as 306a, 306b, 306c, 306d, 306c and 306f). FIG. 3B includes a representation of a portion of the transducer-based device 300 shown in FIG. 3A, but as viewed from a different viewing direction. FIG. 3D includes a representation of a portion of the transducer-based device 300 shown in FIG. 3C, but as viewed from a different viewing direction. It is noted that for clarity of illustration, all the elongate members shown in FIGS. 3C and 3D are not represented in FIGS. 3A and 3B. The plurality of transducers 306 are positionable within a bodily cavity. For example, in some embodiments, the transducers 306 are able to be positioned in a bodily cavity by movement into, within, or into and within the bodily cavity, with or without a change in a configuration of the plurality of transducers 306. In some embodiments, the plurality of transducers 306 are arranged to form a two-or three-dimensional distribution, grid or array of the transducers capable of mapping, ablating or stimulating an inside surface of a bodily cavity or lumen without requiring mechanical scanning. As shown, for example, in FIGS. 3A and 3B, the plurality of transducers 306 are arranged in a distribution receivable in a bodily cavity. In various ones of the FIG. 3, each of at least some of transducers 306 includes a respective electrode 315 (not all of the electrodes 315 called out in each of the FIG. 3, some of the electrodes in FIG. 3D called out as 315a, 315b, 315c, 315d, 315c and 315f).

According to some embodiments, the elongate members 304 may be arranged in a frame or structure 308 that is selectively movable between an unexpanded or delivery configuration (e.g., as shown in FIGS. 3A, 3B) and an expanded or deployed configuration (e.g., as shown in FIGS. 3C, 3D) that may be used during a positioning of the elongate members 304 against a tissue surface within the bodily cavity or during a positioning of the elongate members 304 in the vicinity of the tissue surface. At least the expanded or deployed configuration shown in FIGS. 3C and 3D is an example of a three-dimensional distribution of the transducers 306. In some embodiments, structure 308 has a size in the unexpanded or delivery configuration suitable for delivery through a bodily opening (e.g., via catheter sheath 312 (shown in FIGS. 3A and 3B but removed from FIGS. 3C and 3D for clarity)) to the bodily cavity. At least in a state in which the structure 308 is in the expanded or deployed configuration, the structure 308 may be considered to have two opposing poles 341a and 341b, marked by the intersection with axis 342 extending through the structure 308 as shown in FIGS. 3C and 3D. At least some of the plurality of transducers 306 are circumferentially arranged, e.g., in successive ring-like arrangements, about each of the poles 341a and 341b according to some embodiments. Two such ring-like arrangements are illustrated, for example, as broken-line rings 343a and 343b in FIG. 3C and FIG. 3D, respectively. At least some of the plurality of transducers 306 are arranged in a plurality of groups of the transducers 306, the groups of transducers 306 arranged like lines of longitude (e.g., along respective elongate members 304) about the structure 308 between each of the poles 341a and 341b, according to some embodiments. At least some of the plurality of transducers 306 are arranged in a plurality of groups of the transducers 306, the transducers in each group of transducers 306 arrayed along a path (e.g., along at least a respective portion of a respective elongate member 304) that extends toward the pole 341a, the pole 341b, or both poles 341a and 341b, according to some embodiments. In some embodiments, each path extends like a line of longitude between the poles 341a and 341b.

In various embodiments, catheter sheath 312 typically includes a length sufficient to allow the catheter sheath to extend between a location at least proximate a bodily cavity into which the structure 308 is to be delivered and a location outside a body including the bodily cavity. In some embodiments, structure 308 has a size in the expanded or deployed configuration too large for delivery through a bodily opening (e.g., via catheter sheath 312) to the bodily cavity. The elongate members 304 may form part of a flexible circuit structure (e.g., also known as a flexible printed circuit board (PCB) circuit). The elongate members 304 may include a plurality of different material layers. Each of the elongate members 304 may include a plurality of different material layers. The structure 308 may include a shape memory material, for instance, Nitinol. The structure 308 can include a metallic material, for instance stainless steel, or non-metallic material, for instance polyimide, or both a metallic and non-metallic material by way of non-limiting example. The incorporation of a specific material into structure 308 may be motivated by various factors including the specific requirements of each of the unexpanded or delivery configuration and expanded or deployed configuration, the required position or orientation (e.g., pose), or both of structure 308 in the bodily cavity or the requirements for successful ablation of a desired pattern.

FIG. 4 is a schematic side elevation view of at least a portion of a transducer-based device 400 that includes a flexible circuit structure 401 that is employed to provide a plurality of transducers 406 (two called out) according to an example embodiment. In some embodiments, the flexible circuit structure 401 may form part of a structure (e.g., structure 308) that is selectively movable between a delivery configuration sized for percutaneous delivery and expanded or deployed configurations sized too large for percutaneous delivery. In some embodiments, the flexible circuit structure 401 may be located on, or form at least part of, a structural component (e.g., elongate member 304) of a transducer-based device system.

The flexible circuit structure 401 can be formed by various techniques including flexible printed circuit techniques. In some embodiments, the flexible circuit structure 401 includes various layers including flexible layers 403a, 403b and 403c (e.g., collectively flexible layers 403). In some embodiments, each of flexible layers 403 includes an electrical insulator material (e.g., polyimide). One or more of the flexible layers 403 can include a different material than another of the flexible layers 403. In some embodiments, the flexible circuit structure 401 includes various electrically conductive layers 404a, 404b and 404c (collectively electrically conductive layers 404) that are interleaved with the flexible layers 403. In some embodiments, each of the electrically conductive layers 404 is patterned to form various electrically conductive elements. For example, electrically conductive layer 404a is patterned to form a respective electrode 415 of each of the transducers 406. Electrodes 415 have respective electrode edges 415-1 that form a periphery of an electrically conductive surface associated with the respective electrode 415. It is noted that other electrodes employed in other embodiments may have electrode edges arranged to form different electrodes shapes (for example, as shown by electrode edges 315-1 in FIG. 3C).

Electrically conductive layer 404b is patterned, in some embodiments, to form respective temperature sensors 408 for each of the transducers 406 as well as various leads 410a arranged to provide electrical energy to the temperature sensors 408. In some embodiments, each temperature sensor 408 includes a patterned resistive member 409 (two called out) having a predetermined electrical resistance. In some embodiments, each resistive member 409 includes a metal having relatively high electrical conductivity characteristics (e.g., copper). In some embodiments, electrically conductive layer 404c is patterned to provide portions of various leads 410b arranged to provide an electrical communication path to electrodes 415. In some embodiments, leads 410b are arranged to pass though vias in flexible layers 403a and 403b to connect with electrodes 415. Although FIG. 4 shows flexible layer 403c as being a bottom-most layer, some embodiments may include one or more additional layers underneath flexible layer 403c, such as one or more structural layers, such as a steel or composite layer. These one or more structural layers, in some embodiments, are part of the flexible circuit structure 401 and can be part of, e.g., elongate member 304. In some embodiments, the one or more structural layers may include at least one electrically conductive surface (e.g., a metallic surface) exposed to blood flow. In addition, although FIG. 4 shows only three flexible layers 403a-403c and only three electrically conductive layers 404a-404c, it should be noted that other numbers of flexible layers, other numbers of electrically conductive layers, or both, can be included.

In some embodiments, electrodes 415 are employed to selectively deliver ablation energy (e.g., thermal ablation energy or PFA energy) to various tissue structures within a bodily cavity (e.g., an intra-cardiac cavity or chamber). The energy delivered to the tissue structures may be sufficient for ablating portions of the tissue structures. The energy delivered to the tissue may be delivered to cause monopolar tissue ablation, bipolar tissue ablation or blended monopolar-bipolar tissue ablation by way of non-limiting example.

Energy that is sufficient for tissue ablation may be dependent upon factors including transducer location, size, shape, relationship with respect to another transducer or a bodily cavity, material or lack thereof between transducers, et cetera. Typically, a larger electrode (e.g., an electrode with a relatively large surface area) will achieve a given ablation depth sooner than a smaller electrode. Put differently, a maximum ablation depth of a relatively smaller electrode is typically shallower than that of a relatively larger electrode when ablating under the same control parameters as a relatively larger electrode.

In some embodiments, each electrode 415 is employed to sense or sample an electrical potential in the tissue proximate the electrode 415 at a same or different time than delivering energy sufficient for tissue ablation. In some embodiments, each electrode 415 is employed to sense or sample intra-cardiac voltage data in the tissue proximate the electrode 415. In some embodiments, each electrode 415 is employed to sense or sample data in the tissue proximate the electrode 415 from which an electrogram (e.g., an intra-cardiac electrogram) may be derived. In some embodiments, each resistive member 409 is positioned adjacent a respective one of the electrodes 415. In some embodiments, each of the resistive members 409 is positioned in a stacked or layered array with a respective one of the electrodes 415 to form a respective one of the transducers 406. In some embodiments, the resistive members 409 are connected in series to allow electrical current to pass through all of the resistive members 409. In some embodiments, leads 410a are arranged to allow for a sampling of electrical voltage across each resistive member 409. This arrangement allows for the electrical resistance of each resistive member 409 to be accurately measured. The ability to accurately measure the electrical resistance of each resistive member 409 may be motivated by various reasons including determining temperature values at locations at least proximate the resistive member 409 based at least on changes in the resistance caused by convective cooling effects (e.g., as provided by blood flow).

Referring to FIGS. 3A, 3B, 3C, and 3D transducer-based device 300 can communicate with, receive power from or be controlled by a transducer-activation system 322. In some embodiments, the transducer-activation system 322 represents one or more particular implementations of the system 100 illustrated in FIG. 1. In some embodiments, the transducer-based device 300 or the transducer-based device 200 may be considered part of the transducer-activation system 322 or 100. However, the transducer activation system 322 or 100, according to various embodiments, is not limited to including or interacting with either of the particular transducer-based devices 200, 300, and may include or interact with one or more other types of transducer-based devices in some embodiments.

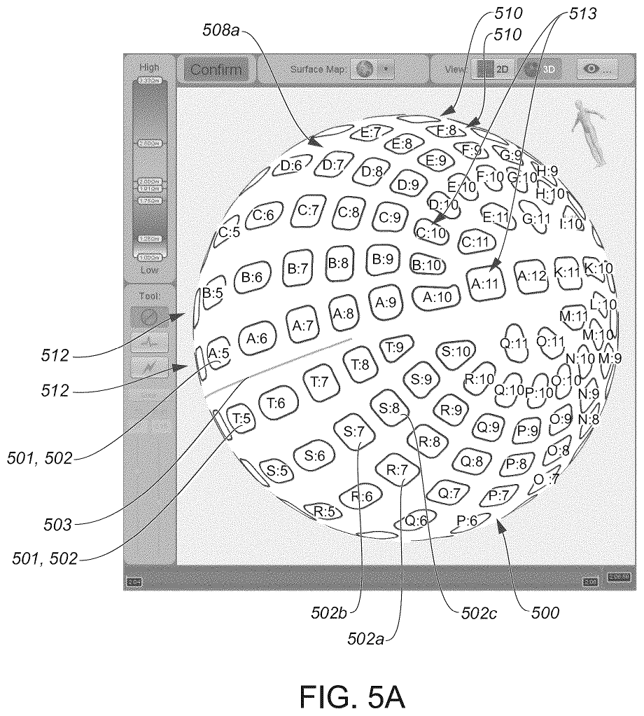

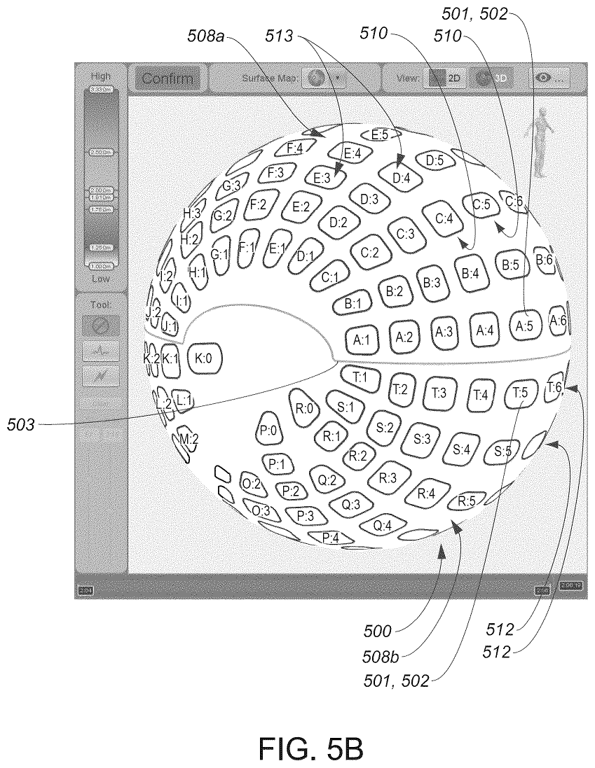

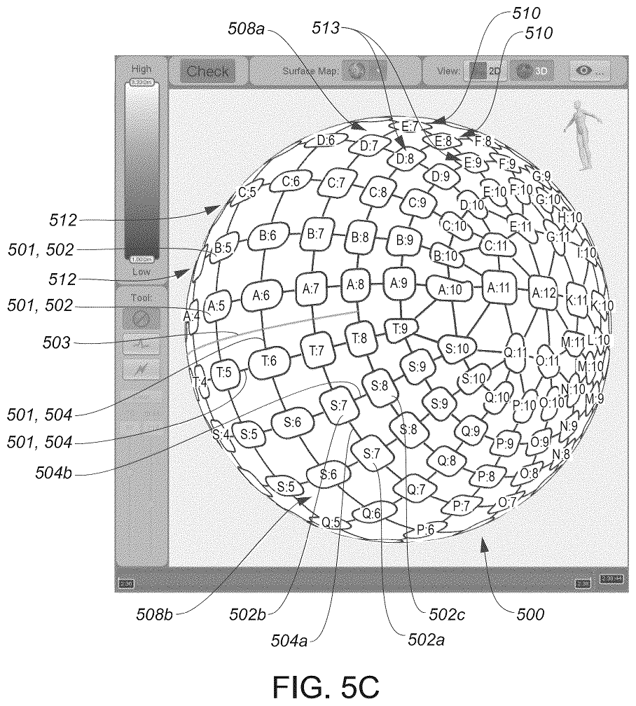

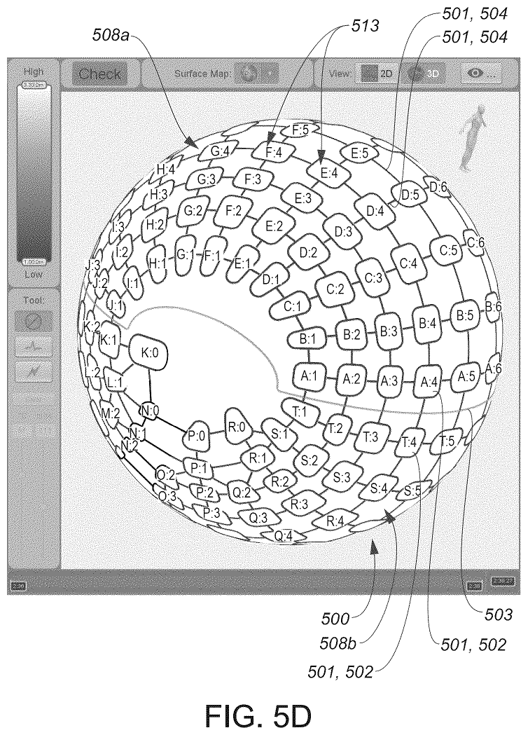

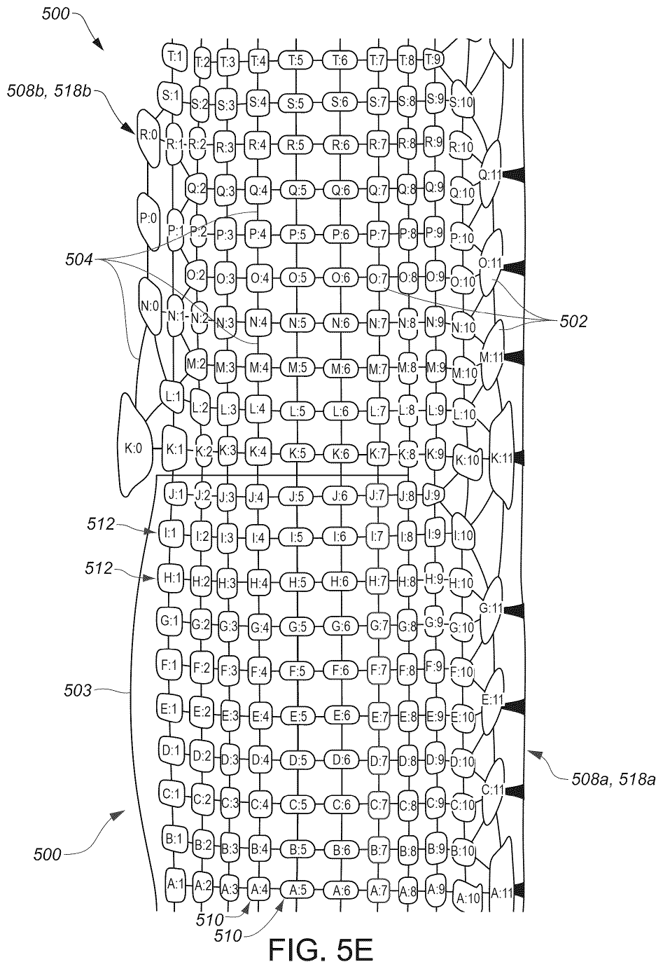

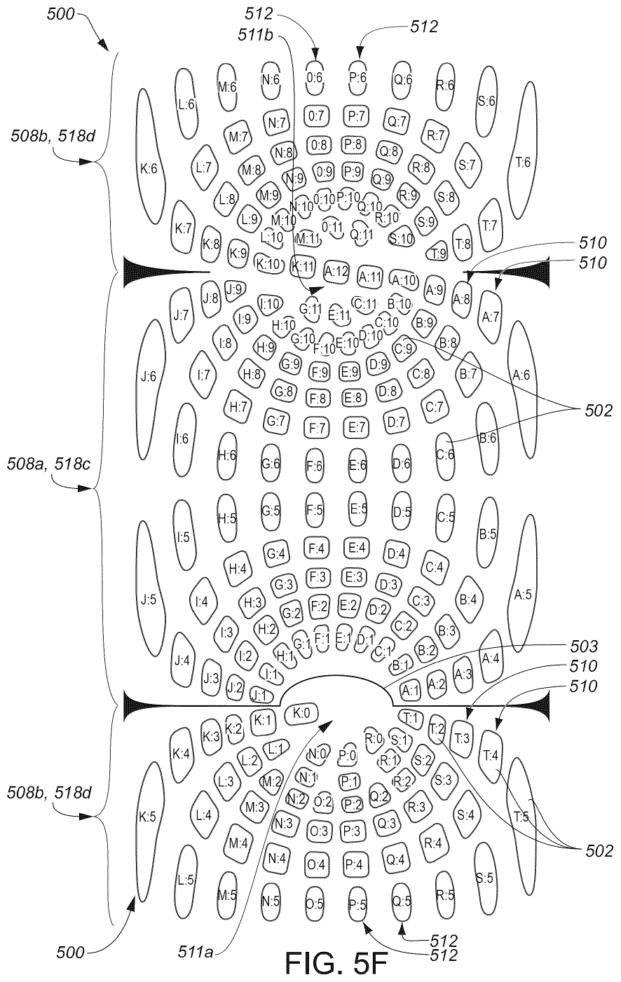

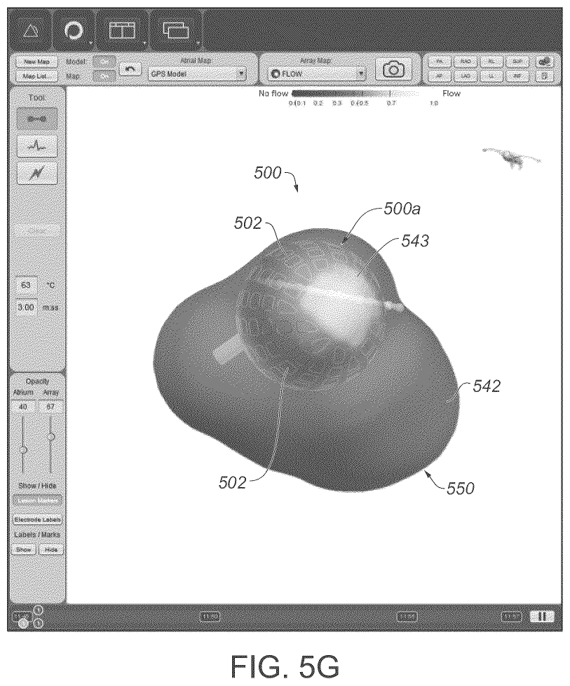

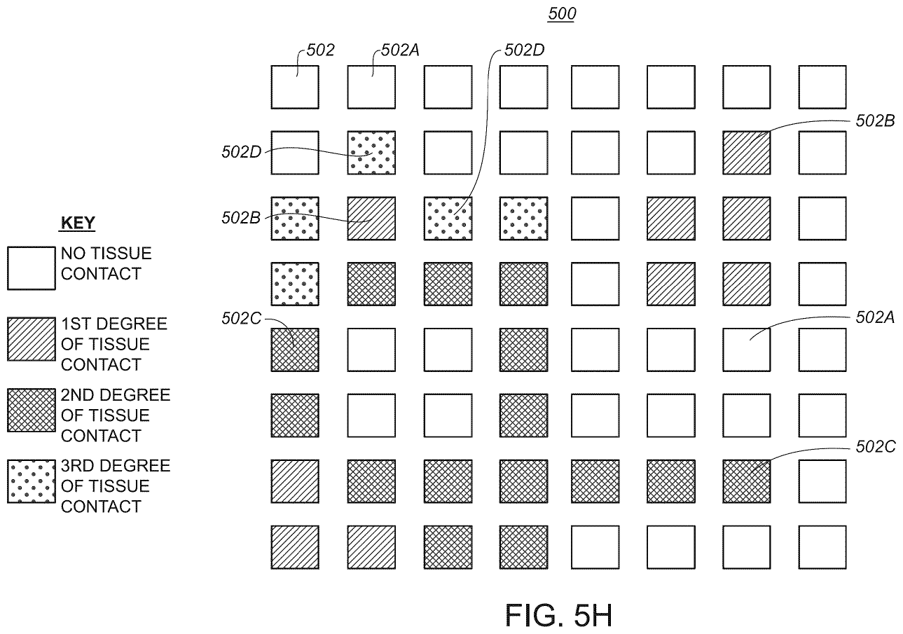

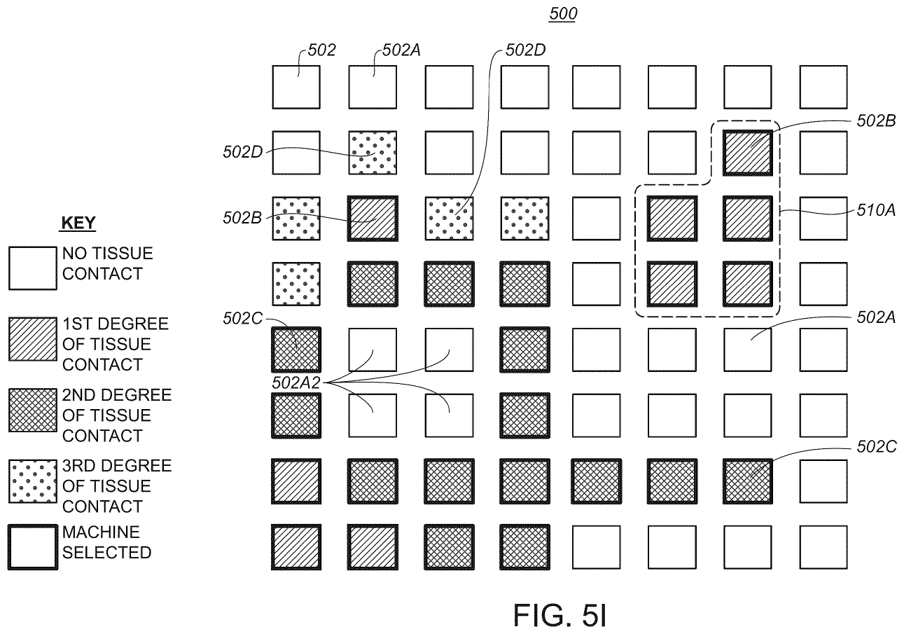

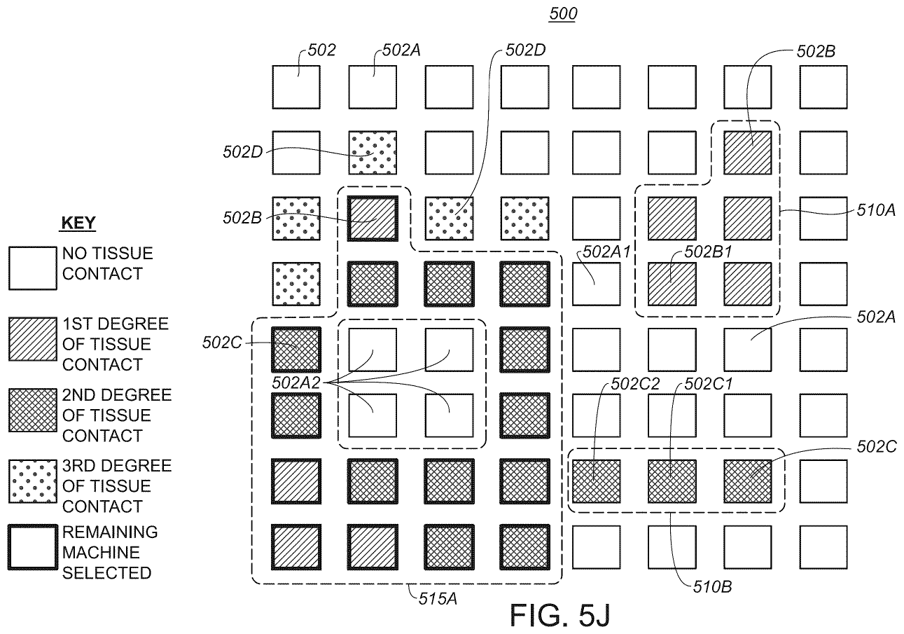

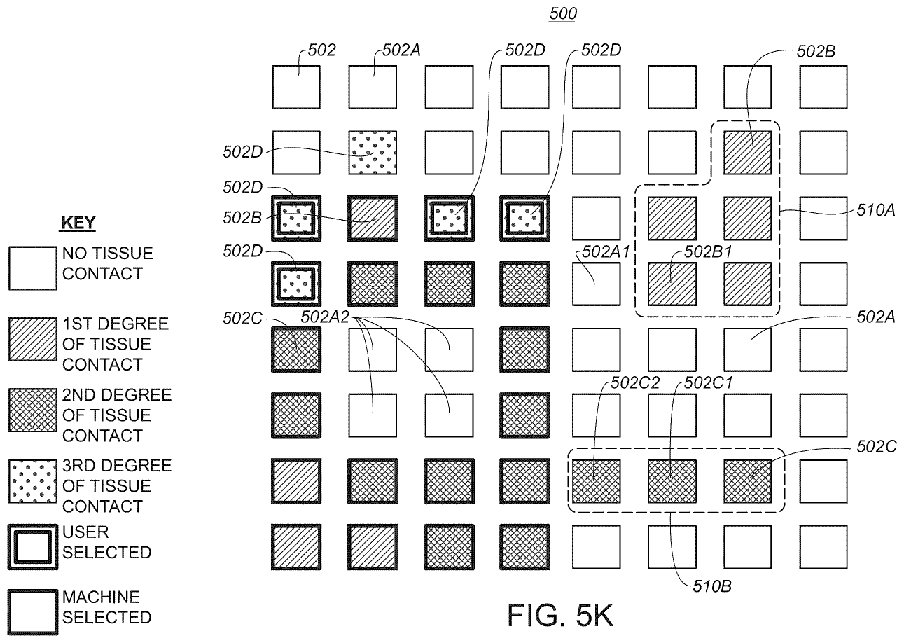

In some embodiments, elongate members 304 can form a portion of an elongated cable 316 of leads 317 (e.g., control leads, data leads, power leads or any combination thereof), for example, by stacking multiple layers, and terminating at a connector 321 or other interface with transducer-activation system 322. The leads 317 may correspond to the electrical connectors 216 in FIG. 2 in some embodiments. The transducer-activation device system 322 may include a controller 324 that includes a data processing device system 310 (e.g., which may be a particular implementation of data processing device system 110 from FIG. 1) and a memory device system 330 (e.g., which may be a particular implementation of the memory device system 130 from FIG. 1) that stores data and instructions that are executable by the data processing device system 310 to process information received from transducer-based device 300 or to control operation of transducer-based device 300, for example, activating various selected transducers 306 to ablate tissue and control a user interface (e.g., of input-output device system 320) according to various embodiments including at least those described below with respect to various ones of FIG. 5. Controller 324 may include one or more controllers.

Transducer-activation device system 322 includes an input-output device system 320 (e.g., which may be a particular implementation of the input-output device system 120 from FIG. 1) communicatively connected to the data processing device system 310 (e.g., via controller 324 in some embodiments). Input-output device system 320 may include a user-activatable control that is responsive to a user action. Input-output device system 320 may include one or more user interfaces or input/output (I/O) devices, for example, one or more display device systems 332, speaker device systems 334, one or more keyboards, one or more mice (e.g., mouse 335), one or more joysticks, one or more track pads, one or more touch screens or other transducers to transfer information to, from, or both to and from a user, for example, a care provider such as a physician or technician. For example, output from a mapping process may be displayed on a display device system 332. Input-output device system 320 may include one or more user interfaces or input/output (I/O) devices, for example, one or more display device systems 332, speaker device systems 334, keyboards, mice, joysticks, track pads, touch screens or other transducers employed by a user to indicate a particular selection or series of selections of various graphical information. Input-output device system 320 may include a sensing device system 325 configured to detect various characteristics including, but not limited to, at least one of tissue characteristics (e.g., electrical characteristics such as tissue impedance, tissue conductivity, tissue type, tissue thickness) and thermal characteristics such as temperature. In this regard, the sensing device system 325 may include one, some, or all of the transducers 306 (or 406 of FIG. 4) of the transducer-based device 300, including the internal components of such transducers shown in FIG. 4, such as the electrodes 415 and temperature sensors 408.

Transducer-activation device system 322 may also include an energy source device system 340 including one or more energy source devices connected to transducers 306. In this regard, although various ones of FIG. 3 show a communicative connection between the energy source device system 340 and the controller 324 (and its data processing device system 310), the energy source device system 340 may also be connected to the transducers 306 via a communicative connection that is independent of the communicative connection with the controller 324 (and its data processing device system 310). For example, the energy source device system 340 may receive control signals via the communicative connection with the controller 324 (and its data processing device system 310), and, in response to such control signals, deliver energy to, receive energy from, or both deliver energy to and receive energy from one or more of the transducers 306 via a communicative connection with such transducers 306 (e.g., via one or more communication lines through catheter body or shaft 314, elongated cable 316 or catheter sheath 312) that does not pass through the controller 324. In this regard, the energy source device system 340 may provide results of its delivering energy to, receiving energy from, or both delivering energy to and receiving energy from one or more of the transducers 306 to the controller 324 (and its data processing device system 310) via the communicative connection between the energy source device system 340 and the controller 324.

The energy source device system 340 may, for example, be connected to various selected transducers 306 to selectively provide energy in the form of electrical current or power (e.g., thermal ablation energy or PFA energy), light or low temperature fluid to the various selected transducers 306 to cause ablation of tissue. The energy source device system 340 may, for example, selectively provide energy in the form of electrical current to various selected transducers 306 and measure a temperature characteristic, an electrical characteristic, or both at a respective location at least proximate each of the various transducers 306. The energy source device system 340 may include various electrical current sources or electrical power sources as energy source devices. In some embodiments, an indifferent electrode 326 is provided to receive at least a portion of the energy transmitted by at least some of the transducers 306. Consequently, although not shown in various ones of FIG. 3, the indifferent electrode 326 may be communicatively connected to the energy source device system 340 via one or more communication lines in some embodiments. In addition, although shown separately in various ones of FIG. 3, indifferent electrode 326 may be considered part of the energy source device system 340 in some embodiments. In various embodiments, indifferent electrode 326 is positioned on an external surface (e.g., a skin-based surface) of a body that comprises the bodily cavity into which at least transducers 306 are to be delivered.

It is understood that input-output device system 320 may include other systems. In some embodiments, input-output device system 320 may optionally include energy source device system 340, transducer-based device 300 or both energy source device system 340 and transducer-based device 300 by way of non-limiting example. Input-output device system 320 may include the memory device system 330 in some embodiments.

Structure 308 can be delivered and retrieved via a catheter member, for example, a catheter sheath 312. In some embodiments, a structure provides expansion and contraction capabilities for a portion of the medical device (e.g., an arrangement, distribution or array of transducers 306). The transducers 306 can form part of, be positioned or located on, mounted or otherwise carried on the structure and the structure may be configurable to be appropriately sized to slide within catheter sheath 312 in order to be deployed percutaneously or intravascularly. FIGS. 3A, 3B show one embodiment of such a structure. In some embodiments, each of the elongate members 304 includes a respective distal end 305 (only one called out in each of FIGS. 3A, 3B), a respective proximal end 307 (only one called out in each of FIGS. 3A, 3B) and an intermediate portion 309 (only one called out in each of FIGS. 3A, 3B) positioned between the proximal end 307 and the distal end 305. The respective intermediate portion 309 of each elongate member 304 includes a first or front surface 318a that is positionable to face an interior tissue surface within a bodily cavity and a second or back surface 318b opposite across a thickness of the intermediate portion 309 from the front surface 318a. In some embodiments, each of the elongate members 304 is arranged front surface 318a—toward-back surface 318b in a stacked array during an unexpanded or delivery configuration similar to that described in International Publication No.: WO 2012/100184, published Jul. 26, 2012 and International Publication No.: WO 2012/100185, published Jul. 26, 2012. In many cases a stacked array allows the structure 308 to have a suitable size for percutaneous or intravascular delivery. In some embodiments, the elongate members 304 are arranged to be introduced into a bodily cavity distal end 305 first. A flexible, elongated, catheter body 314 is used to deliver structure 308 through catheter sheath 312 according to some embodiments.

In a manner similar to that described in International Publication No.: WO 2012/100184, published Jul. 26, 2012 and International Publication No.: WO 2012/100185, published Jul. 26, 2012, each of the elongate members 304 is arranged in a fanned arrangement 370 in FIGS. 3C, 3D. In some embodiments, the fanned arrangement 370 is formed during the expanded or deployed configuration in which structure 308 is manipulated to have a size too large for percutaneous or intravascular delivery. In some embodiments, structure 308 includes a proximal portion 308a having a first domed shape 309a and a distal portion 308b having a second domed shape 309b. In some embodiments, the proximal and the distal portions 308a, 308b each include respective portions of elongate members 304. In some embodiments, the structure 308 is arranged to be delivered distal portion 308b first into a bodily cavity when the structure is in the unexpanded or delivery configuration as shown in FIGS. 3A, 3B. In various embodiments, the proximal and distal portions 308a, 308b do not include a domed shape in the delivery configuration (for example, as shown in FIGS. 3A, 3B). In some embodiments, the first domed shape 309a of the proximal portion 308a and the second domed shape 309b of the distal portion 308b are arranged in a clam shell configuration in the expanded or deployed configuration shown in FIGS. 3C, 3D.

The transducers 306 can be arranged in various distributions or arrangements in various embodiments. In some embodiments, various ones of the transducers 306 are spaced apart from one another in a spaced apart distribution in the delivery configuration shown in FIGS. 3A, 3B. In some embodiments, various ones of the transducers 306 are arranged in a spaced apart distribution in the deployed configuration shown in FIGS. 3C, 3D. In some embodiments, various pairs of transducers 306 are spaced apart with respect to one another. In some embodiments, various spaces are located between various pairs of the transducers 306. For example, in FIG. 3D the transducer-based device 300 includes at least a first transducer 306a, a second transducer 306b and a third transducer 306c (all collectively referred to as transducers 306). In some embodiments each of the first, the second and the third transducers 306a, 306b and 306c are adjacent transducers in the spaced apart distribution (e.g., first transducer 306a is adjacent second transducer 306b, second transducer 306b is adjacent third transducer 306c, and first transducer 306a is adjacent third transducer 306c). In some embodiments, the first and the second transducers 306a, 306b are located on different elongate members 304 while the second and the third transducers 306b, 306c are located on a same elongate member 304. In some embodiments, a first space 350 is between the first and the second transducers 306a, 306b. In various embodiments, a first space 350 is between the respective electrodes 315a, 315b of the first and the second transducers 306a, 306b. In some embodiments, the first space 350 is not associated with any physical portion of structure 308. In some embodiments, a second space 360 associated with a physical portion of device 300 (e.g., a portion of an elongate member 304) is between the second and the third transducers 306b, 306c. In various embodiments, the second space 360 is between the respective electrodes 315b, 315c of the second and the third transducers 306b, 306c. In some embodiments, each of the first and the second spaces 350, 360 does not include a transducer of transducer-based device 300. In some embodiments, each of the first and the second spaces 350, 360 does not include any transducer. It is noted that other embodiments need not employ a group of elongate members 304 as employed in the illustrated embodiment. For example, other embodiments may employ a structure having one or more surfaces, at least a portion of the one or more surfaces defining one or more openings in the structure. In these embodiments, a space not associated with any physical portion of the structure may extend over at least part of an opening of the one or more openings.

In other example embodiments, other structures besides those shown in FIGS. 2 and 3A-3D may be employed to support or carry transducers of a transducer-based device such as a transducer-based catheter. For example, an elongated catheter member may be used to distribute the transducers in a linear or curvilinear array. Basket catheters or balloon catheters may be used to distribute the transducers in a two-dimensional or three-dimensional array.

FIGS. 6A and 6B include respective data generation and flow diagrams, which may implement various embodiments of methods 600A and 600B by way of associated computer-executable instructions according to some example embodiments. In various example embodiments, a memory device system (e.g., memory device systems 130, 330) is communicatively connected to a data processing device system (e.g., data processing device systems 110 or 310, otherwise stated herein as “e.g., 110, 310”) and stores a program executable by the data processing device system to cause the data processing device system to execute various embodiments of methods 600 via interaction with at least, for example, a transducer-based device (e.g., transducer-based devices 200, 300, or 400, in some embodiments). In these various embodiments, the program may include instructions configured to perform, or cause to be performed, various ones of the instructions associated with execution of various embodiments of methods 600A and 600B. In some embodiments, the methods 600A and 600B may include a subset of the associated blocks or additional blocks other than those shown in FIGS. 6A and 6B. In some embodiments, one or both of the methods 600A, 600B may include a different sequence indicated between various ones of the associated blocks shown in FIGS. 6A and 6B. The features recited by any block are not intended to be exclusive, and the adopting of any recited features of any particular block in a particular embodiment does not prevent the inclusion of any other features, according to some embodiments, unless the features cannot work together (i.e., are mutually exclusive).

According to some embodiments, methods 600A and 600B may include block 602 associated with computer-executable instructions (e.g., graphical representation instructions (which also may be referred to at least as graphical interface instructions or display instructions) provided by a program) configured to cause an input-output device system (e.g., 120, 320) to display a graphical representation. Blocks 602a and 602b represent some possible aspects of the graphical representation, according to some embodiments, although either or both of such blocks 602a, 602b need not be included in some embodiments, and one or more other aspects of the graphical representation may be provided in some embodiments. In some embodiments associated with block 602a, the graphical representation includes a graphical representation of at least a portion of a transducer-based device (e.g., 200, 300, or 400 in some embodiments). For example, at least FIG. 5G, discussed in more detail below, illustrates a graphical representation 543 of a transducer-based device, similar to transducer-based device 300. In some embodiments, the graphical representation includes a plurality of graphical elements associated with or corresponding to a plurality of transducers positionable in a bodily cavity. For example, at least FIG. 5G, discussed in more detail below, illustrates a graphical representation 500 including graphical elements 502 (two called out in FIG. 5G) associated with a plurality of transducers of the illustrated transducer-based device. In some embodiments, the graphical representation (e.g., graphical representation 500) includes a plurality of graphical elements (e.g., graphical elements 502) corresponding to a plurality of transducers positionable in a bodily cavity. In some embodiments, each of the plurality of graphical elements may correspond to a respective at least one of the plurality of transducers. According to various embodiments, the transducers of the plurality of transducers may be individually selectable. According to various embodiments, the transducers of the plurality of transducers may be individually addressable. According to various embodiments, the transducers of the plurality of transducers may be individually activatable. In some embodiments associated with block 602b in FIG. 6A, the graphical representation includes a representation of at least a portion of a bodily cavity. For example, at least FIG. 5G, discussed in more detail below, illustrates a graphical representation 542 of at least a portion of a bodily cavity, which is an atrium in the case of FIG. 5G.

FIG. 5A illustrates a graphical interface including a graphical representation 500 provided by the input-output device system according to some embodiments provided in accordance with graphical representation instructions associated with block 602 or block 602a in FIG. 6A. In some embodiments, the graphical representation 500 includes a three-dimensional graphical representation of at least a portion of a transducer-based device (e.g., like structure 308 in at least FIG. 3D).

The instructions associated with block 602 or block 602a may be configured to access a predefined model (e.g., a computer-aided-design (“CAD”) or other computer-readable model stored in memory device system 130, 330) of the at least the portion of the transducer-based device (e.g., the plurality of transducers) and display the at least the portion of the transducer-based device according to such model. In some embodiments encompassing at least FIG. 5A, a representation of the transducer-based device is provided by various elements of graphical representation 500. In some embodiments, the graphical interface depicts the transducer-based device as including a first domed portion 508a associated with a first domed portion of the transducer-based device (e.g., proximal portion 308a when having the first domed shape 309a) and a second domed portion 508b associated with a second domed portion of the transducer-based device (e.g., distal portion 308b having the second domed shape 309b). A separation graphical element 503 may be employed between the first and the second domed portions 508a, 508b in some embodiments, but may be omitted in other embodiments. Various other transducer-based devices may be depicted according to the instructions associated with block 602 in other embodiments.

In some embodiments, a plurality of graphical elements 501 (only two called out in FIG. 5A) are depicted (e.g., according to the instructions associated with block 602 or 602a, for example, in the case of FIG. 5A) among various elements of graphical representation 500. In various embodiments, each of the graphical elements 501 is respectively associated with a respective one of a plurality of transducer sets. According to some embodiments, each respective transducer set includes at least one transducer of a plurality of transducers included as part of the transducer-based device (e.g., transducer-based devices 200, 300, or 400, according to some embodiments), and each respective transducer set has at least one different transducer than another of the other transducer sets. In various particular embodiments, each respective transducer set has at least one different transducer than each of the other transducer sets.

FIG. 5B shows the graphical interface in which the display instructions (e.g., associated with block 602, 602a) have been configured to cause (for example, in response to user input via an input-output device system such as 120, 320) the three-dimensional graphical representation of the transducer-based device to be manipulated so as to be viewed from a different viewing angle than that shown in FIG. 5A. It is noted that three-dimensional representations of at least a portion of a transducer-based device are shown in FIGS. 5A, 5B, 5C, 5D, and 5G, according to some embodiments. Two-dimensional representations of at least a portion of a transducer-based device are shown in FIGS. 5E, 5F, 5H, 5I, 5J, and 5K, according to some embodiments.

Referring to some embodiments encompassing FIG. 5A, each of at least some of the graphical elements 501 is provided by a respective one of a plurality of transducer graphical elements 502 that include at least a first transducer graphical element 502a, a second transducer graphical element 502b, and a third transducer graphical element 502c (e.g., all the transducer graphical elements forming part of a group of transducer graphical elements 502). In some embodiments, each transducer graphical element 502 is associated with, or corresponds to, a single respective transducer (e.g., a transducer 220, 306, or 406 in some embodiments) of the transducer-based device (e.g., transducer-based device 200, 300, or 400, according to some embodiments). In some example embodiments, each transducer graphical element 502 is representative of a respective transducer (e.g., a transducer 220, 306, or 406, in some embodiments) of the transducer-based device (e.g., 200, 300, or 400, according to some embodiments). However, in some embodiments, multiple graphical elements may be associated with, or correspond to, a single respective transducer of the transducer-based device. In some embodiments, each transducer may be identified by one or more graphical elements that do not represent the physical appearance of the transducer. For instance, a graphical user interface may include a text-based identifier, such as “A:1” to identify to a user a transducer in a first row and a first column in a grid of transducers, or “B:2” to identify to the user a transducer in a second row and a second column in a grid of transducers. In some example embodiments, each transducer graphical element 502 is representative of a location or position of a respective transducer of the transducer-based device. In some embodiments, the graphical representation 500 includes a first spatial relationship between the transducer graphical elements 502 that is consistent with a second spatial relationship between the corresponding transducers associated with the transducer graphical elements 502. For example, in some embodiments, the transducer graphical elements 502 in the three-dimensional graphical representation 500 in FIGS. 5A, 5B may exhibit a same spatial relationship that the transducers 306 exhibit in the transducer-based device 300 in FIGS. 3C and 3D. Or, in some embodiments, the transducer graphical elements 502 in other graphical representations 500 in others of FIG. 5 may exhibit a respective or corresponding spatial relationship that the transducers 306 exhibit in the respective transducer-based device. In this regard, in some embodiments, the graphical representation 500 may include a first spatial relationship between the transducer graphical elements 502 that is consistent with a second spatial relationship between the corresponding transducers associated with the transducer graphical elements 502 when the corresponding transducers are arranged in a deployed configuration (e.g., such as FIGS. 3C and 3D). For example, an adjacent pair of transducers (e.g., an adjacent pair of first transducer 306a and second transducer 306b, an adjacent pair of second transducer 306b and third transducer 306c, or an adjacent pair of first transducer 306a and third transducer 306c) arranged according to the second spatial relationship may correspond to an adjacent pair of the transducer graphical elements 502 arranged according to the first spatial relationship. In some embodiments, each particular depicted transducer graphical element 502 is shown having a shape that is consistent with the particular transducer (or portion thereof) that the particular transducer graphical element 502 is representative of.

In some example embodiments, graphical elements 501 may include alternate or additional forms. For example, FIG. 5C shows an example embodiment in which each of at least some of the graphical elements 501 are provided by a respective one of a plurality of between graphical elements 504 including a first between graphical element 504a and a second between graphical element 504b (e.g., all the between graphical elements collectively referred to as between graphical elements 504). The graphical elements are referred to as “between graphical elements” since they may be respectively located between, e.g., a respective group, or pair, in some embodiments, of adjacent transducer graphical elements 502, according to some embodiments. FIG. 5D shows an embodiment of the graphical interface in which the display instructions have been configured to cause (for example, in response to a user input via input-output device system 120 or 320) the depiction of the transducer-based device to be manipulated so as to be viewed from a different viewing angle than that shown in FIG. 5C.

In some embodiments, between graphical elements 504 are shown in addition to various ones of the transducer graphical elements 502 shown in FIGS. 5A and 5B. In some embodiments, between graphical elements 504 are provided separately or with other embodiments of graphical elements 501. In various embodiments, each of the between graphical elements 504 is associated with a set of at least two (e.g., a group) of the transducers of the transducer-based device. In some example embodiments, each of the between graphical elements 504 is associated with a pair of transducers in the transducer-based device. In some example embodiments, each between graphical element 504 is associated with a space between a respective pair of transducers in the transducer-based device. In some example embodiments, each between graphical element 504 is associated with a space between a respective pair of adjacent ones of the transducers in the transducer-based device. In some example embodiments, each between graphical element 504 is associated with a space between a respective pair of adjacent ones of the transducer graphical elements 502 in the graphical representation 500.

In some embodiments, selection of a between graphical element 504 may allow an efficient selection of multiple transducer graphical elements 502. For example, with reference to FIG. 5C, if between graphical element 504a is selected by a user interacting with the graphical interface of FIG. 5C, such selection may cause adjacent transducer graphical elements 502a and 502b to be selected. In some embodiments associated with FIG. 5C, if between graphical element 504a is selected by a user interacting with the graphical interface of FIG. 5C, such selection may cause adjacent transducer graphical elements 502a and 502b to be concurrently selected. In some embodiments, such a selection of a between graphical element 504 may cause selection of the corresponding pair of transducer graphical elements 502 for bipolar ablation performed by the physical transducers (e.g., transducers 220, 306, or 406) corresponding to the pair of transducer graphical elements 502. In some embodiments, such a selection of a between graphical element 504 may cause selection of the corresponding pair of transducer graphical elements 502 for concurrent activation of the physical transducers (e.g., transducers 220, 306, or 406) corresponding to the pair of transducer graphical elements 502. In this regard, a single transducer may be associated with multiple graphical elements, according to some embodiments. For instance, with respect to FIG. 5C, the physical transducer 306 that corresponds to transducer graphical element 502a may be associated not only with transducer graphical element 502a, but also each of its connected between graphical elements 504, in some embodiments. Further, although various ones of FIGS. 5 illustrate a one-to-one correspondence between transducer graphical element 502 and transducer 306, such a one-to-one correspondence is not necessary in some embodiments. For instance, a transducer 306 may be represented by a symbol or other graphical depiction, which may be represented as a cluster of graphical elements, such cluster being considered to represent the single transducer 306, in some embodiments.

In some embodiments, first transducer graphical element 502a (e.g., FIG. 5C) is associated with, or corresponds to, a first transducer (e.g., first transducer 306a, e.g., FIG. 3D) of the transducer-based device, second transducer graphical element 502b is associated with, or corresponds to, a second transducer (e.g., second transducer 306b) of the transducer-based device, and third transducer graphical element 502c is associated with, or corresponds to, a third transducer (e.g., third transducer 306c) of the transducer-based device. In some embodiments, the first between graphical element 504a is associated with a first space that is between the first and the second transducers, and the second between graphical element 504b is associated with a second space that is between the second and the third transducers. In some embodiments, the first space is a space that is not associated with any physical part of the transducer-based device (e.g., first space 350) and the second space is a space that is associated with a physical part of the transducer-based device (e.g., second space 360). In some embodiments, each of the first and the second between graphical elements 504a, 504b is associated with a space that does not include a transducer of the transducer-based device. In some embodiments, each of the first and the second between graphical elements 504a, 504b is associated with a space that does not include any transducer. It is understood that a “space” or “region of space” need not be a vacant space but can include physical matter therein.

In some embodiments, the first between graphical element 504a is positioned between the second and the first transducer graphical elements 502b, 502a among the graphical representation 500. In some embodiments, the second between graphical element 504b is positioned between the second and the third transducer graphical elements 502b, 502c among the graphical representation 500. In some embodiments, the second and the first transducer graphical elements 502b, 502a are adjacent transducer graphical elements. In some embodiments, the second and the first transducer graphical elements 502b, 502a are associated with adjacent transducers (e.g., transducers 306b, 306a). In some embodiments, the second and the third transducer graphical elements 502b, 502c are adjacent transducer graphical elements. In some embodiments, the second and the third transducer graphical elements 502b, 502c are associated with adjacent transducers (e.g., transducers 306b, 306c). In some embodiments, the first and the third transducer graphical elements 502a, 502c are adjacent transducer graphical elements. In some embodiments, the first and the third transducer graphical elements 502a, 502c are associated with adjacent transducers (e.g., transducers 306a, 306c). In other example embodiments, other spatial relationships exist between the transducer graphical elements 502 and the between graphical elements 504 in the graphical representation 500.

The transducer graphical elements 502, the between graphical elements 504, or both may have different sizes, shapes or forms than those shown in the illustrated embodiments. In some embodiments, at least one particular one of the transducer graphical elements 502 may be depicted with a different shape, size, or form than the respective one of the shape, size, or form of the respective portion of the particular transducer to which the particular one of the transducer graphical elements 502 corresponds. In some embodiments, different ones of the between graphical elements 504 may be depicted with different shapes, sizes, or forms.

With reference to various ones of FIGS. 5, at least a portion of the transducer graphical elements 502 and at least a portion of the between graphical elements 504 are arranged in a plurality of rows 510 (two called out in FIG. 5C) and a plurality of columns 512 (two called out in FIG. 5C). In some embodiments, each row 510 corresponds to a respective one of number “0”, “1”, “2”, “3”, “4”, “5”, “6”, “7”, “8”, “9”, “10”, and “11”, and each column 512 corresponds to a respective one of letters “A”, “B”, “C”, “D”, “E”, “F”, “G”, “H”, “I”, “J”, “K”, “L”, “M”, “N”, “O”, “P”, “Q”, “R”, “S”, and “T”, each of the numbers and letters used as part of the unique identifier 513 (only two called out with reference numeral 513 in FIG. 5C with respect to D:8 and E:9) of each transducer graphical element 502. In some embodiments, the plurality of rows 510 and columns 512 correspond to a condition in which structure 308 is in the deployed configuration. In some embodiments, a portion of each of the columns 512 may correspond to a space associated with a physical portion of the transducer-based device (e.g., an elongate member 304). In some embodiments, each of the columns 512 may correspond to at least a portion of the transducers located on a particular elongate member (e.g., an elongate member 304) of a transducer-based device. In some embodiments, at least one of the columns 512 may include at least one transducer graphical element 502 having a shape that is different than the respective shape comprised by any of the transducer graphical elements 502 included in at least one other of the columns 512. For example, the “A” column 512 includes a transducer graphical element 502 identified as “A:10” that has a shape that is different than any of the transducer graphical elements 502 comprised by at least one of the other columns 512. In some embodiments, at least a first one of the rows 510 may include identically shaped transducer graphical elements 502 (e.g., row 510 that includes transducer graphical elements 502 identified as “A:6”, “B:6”,“C:6”, “D:6”, “E:6”, “F:6”, “G:6”, “H:6”, “I:6”, “J:6”, “K:6”, “L:6”, “M:6”, “N:6”, “O:6”, “P:6”, “Q:6”, “R:6”, “S:6”, and “T:6” (not all of which are shown in FIG. 5A due to the presented perspective)), and at least a second one of the rows 510 may include differently shaped transducer graphical elements 502 (e.g., row 510 that includes transducer graphical elements 502 identified as “A:10”, “B:10”, “C:10”, “D:10”, “E:10”, “F:10”, “G:10”, “H:10”,“I:10”, “K:10”, “L:10”, “M:10”, “N:10”, “O:10”, “P:10”, “Q:10”, “R:10”, and “S:10”).

In some example embodiments, a portion of each of the rows 510 may correspond to spaces not associated with any physical portion of the transducer-based device (e.g., space 350 between adjacent ones of the elongate members 304). In other example embodiments, different numbers of transducer graphical elements 502 and different numbers and spatial arrangements of between graphical elements 504 may be depicted in the graphical representation. In other example embodiments, different numbers and spatial arrangements of rows 510 and columns 512 may be depicted in the graphical representation. In various embodiments, each of the between graphical elements (e.g., between graphical elements 504) depicted in the graphical representation may be representative of a respective physical path extending between a respective pair of transducers of the transducer-based device. Each of the physical paths may extend over a physical surface of the transducer-based device or over a portion of an opening defined by a physical surface of the transducer-based device. In some embodiments like FIG. 5C, each between graphical element 504 may be representative of a respective path in real world space, the path extending (e.g., as the crow flies in some embodiments) between the respective transducers associated with the adjacent pair of transducer graphical elements 502 that the between graphical element 504 extends between. In some embodiments like FIG. 5C, each adjacent pair of the transducer graphical elements 502 may be provided along a row 510 (two called out in FIG. 5C) of the graphical elements 501, along a column 512 (two called out in FIG. 5C) of the graphical elements 501, or diagonally between a row 510 and a column 512.

Referring back to FIGS. 5A, 5B, the plurality of rows 510 and the plurality of columns 512 are depicted as a three-dimensional arrangement in the graphical representation. In some embodiments, at least two of the plurality of columns 512 are depicted in the graphical representation extending along respective directions that converge with respect to one another. In some embodiments, at least two of the plurality of columns 512 are depicted in the graphical representation extending along non-parallel directions and at least two of the plurality of rows 510 are depicted extending along parallel directions. In some embodiments, the rows 510 and the columns 512 are depicted in the graphical representation in an arrangement in which the columns 512 are circumferentially arranged. In some embodiments, the rows 510 and the columns 512 are depicted in the graphical representation in an arrangement having a generally spherical shape. The plurality of columns 512 may be depicted like lines of longitude, and the plurality of rows 510 may be depicted like lines of latitude. Although the rows 510 and columns 512 are illustrated in FIGS. 5A-5D as circumferential lines (like lines of longitude and latitude), such rows 510 and columns 512 can take other forms, as shown, for example, in FIGS. 5E and 5F, discussed in more detail below, according to some embodiments.

The graphical representation instructions associated with block 602 may include instructions (e.g., instructions responsive to a user input made via an input-output device system) configured to vary the depiction of the portion of the transducer-based device between a three-dimensional representation (e.g., as depicted in various ones of at least FIGS. 5A, 5B, 5C, and 5D, according to some embodiments) and a two-dimensional representation (e.g., as depicted by at least FIG. 5E or 5F, according to some embodiments). Various two-dimensional representations are possible in various embodiments. For instance, the plurality of transducer graphical elements 502 may be arranged in the graphical representation 500 in a particular spatial distribution representing the three-dimensional distribution of transducers (e.g., 220 or 306) distorted onto a two-dimensional plane to form the two-dimensional representation. In this regard, in some embodiments, the two-dimensional representation of the three-dimensional distribution of transducers (e.g., 220 or 306) distorted onto a two-dimensional plane is not merely an isometric or other perspective view of the three-dimensional distribution of transducers, as such an isometric or other perspective view would be considered a three-dimensional representation, such as that shown in various ones of FIGS. 5A, 5B, 5C, and 5D. The two-dimensional representation may be generated according to the display instructions according to a conformal map or projection, such as a Mercator map or projection, a transverse Mercator map or projection, or other three-dimensional-to-two-dimensional map or projection, known in the art, according to some embodiments. According to various embodiments, a conformal mapping is a function that preserves local angles. For example, according to some embodiments, when a particular spatial relationship between the plurality of transducers 306 is conformally mapped to the graphical representation 500, an angle defined between a group of transducers (e.g., 306) according to the particular spatial relationship is preserved between the corresponding group of transducer graphical elements 502. In some embodiments, the two-dimensional representation need not be a projection or map from a three-dimensional model, and may merely be any two-dimensional representation, e.g., including an arrangement of transducers.

The two-dimensional representation depicted in FIG. 5E, according to some embodiments, represents the first domed portion 508a (e.g., shown in FIGS. 5C, 5D) of the depicted transducer-based device as first Mercator projection 518a and the second domed portion 508b (e.g., shown in FIGS. 5C, 5D) of the depicted transducer-based device as a second Mercator projection 518b. The first and the second Mercator projections 518a and 518b advantageously allow for simultaneous viewing of all the transducer graphical elements 502 and the between graphical elements 504. Columns 512 and rows 510 are depicted two-dimensionally in FIG. 5E. In some embodiments, separation graphical element 503 is also depicted in a two-dimensional configuration.

As discussed above, other two-dimensional representations may be implemented and may be user-selectable for viewing. For example, FIG. 5F illustrates a transverse Mercator projection employed according to some embodiments. In FIG. 5F, the transverse Mercator projection includes two portions 518c, 518d, each of the portions 518c, 518d representative of a respective one of first and second domed portions 508a and 508b in the corresponding three-dimensional representation. In FIG. 5F, portion 518d of the transverse Mercator projection is shown as two parts, each part at least depicting the transducer graphical elements 502 in a respective one of two parts of the domed portion 508b. In FIG. 5F, portion 518c is representative of first domed portion 508a. In some embodiments, various ones of the columns 512 radiate outwardly radially or quasi-radially from particular ones of a plurality of pole regions 511a and 511b represented in the graphical representation 500. In some embodiments, various ones of the rows 510 are circumferentially arranged about particular ones of a plurality of pole regions 511a and 511b.

In some embodiments, at least some of the between graphical elements 504 are not shown in various ones of the displayable two-dimensional representations. For example, in FIG. 5F, between graphical elements 504 have been selectively controlled, e.g., in response to user input, to be hidden among the graphical representation. In various embodiments, the transducer graphical elements 502 shown in each of the FIGS. 5E and 5F are arranged with respect to one another according to a spatial relationship that corresponds to a spatial relationship that the transducer graphical elements are arranged in the three-dimensional representations shown in various ones of FIGS. 5A, 5B, 5C, and 5D. In various embodiments, the transducer graphical elements 502 shown in each of the FIGS. 5E and 5F are arranged with respect to one another according to a spatial relationship that corresponds to a spatial relationship that particular transducers, which the transducer graphical elements 502 correspond to, are arranged with respect to one another when a supporting structure (e.g., structure 308) is in a deployed configuration.

Various computer-executable instructions may be configured to control various input element control functions (e.g., mouse drag functions, touch screen drag functions) between various operating modes such as rotating and panning modes. A rotating mode may be advantageously used for manipulation of a three-dimensional representation of a transducer-based device or other portions of the graphical representation 500 to allow for viewing one or more portions of the three-dimensional representation of the transducer-based device or various portions of the graphical representation 500 that were not previously viewable (e.g., a manipulation between the views shown in FIGS. 5A and 5B or a manipulation between the views shown in FIGS. 5C and 5D). In some embodiments, a panning mode or rotating mode may be advantageously used for manipulation of a two-dimensional representation of the transducer-based device or other portions of the graphical representation 500 to allow for viewing of different arrangements of various graphical elements in the representation of a transducer-based device or other portions of the graphical representation 500.

The graphical representation 500 displayed according to the instructions associated with block 602 in FIGS. 6A and 6B is not limited to three-dimensional representations of a spatial distribution of the plurality of graphical elements 501 in the context of a display of a three-dimensional representation of a transducer-based device (e.g., as shown in FIGS. 5A, 5B, 5C, and 5D). Other entities may also be graphically depicted in a three-dimensional (or two-dimensional) manner. For example, FIG. 5G illustrates a graphical interface including a graphical representation 500 provided by the input-output device system 120, 320, according to some embodiments provided in accordance with graphical representation instructions associated with block 602 in FIG. 6A. In some embodiments, the graphical representation 500 in FIG. 5G includes a three-dimensional graphical representation of at least a portion of a transducer-based device 500a that is similar to that depicted in FIGS. 5A, 5B, 5C, and 5D. The graphical representation 500 of FIG. 5G also shows, e.g., according to program instructions associated with block 602b in FIG. 6A in some embodiments, a three-dimensional representation of an envelope 550 that graphically represents at least a portion of a bodily cavity (e.g., the atrium of a heart in this depicted embodiment) in which the transducer-based device may be deployed. According to various embodiments, envelope 550 may be generated with the use of a catheter-device-location tracking system or navigation system (e.g., an electro-potential-based navigation system or an electromagnetic-based tracking system). Some examples of such a location tracking system or navigation system can be found at least in U.S. Patent Application Publication No. 2021/0353370, published Nov. 18, 2021. The catheter-device-location tracking system may, in some embodiments, be configured to provide location information derived from a plurality of location signal sets provided to the data processing device system 110, 310. According to various embodiments, the location information may indicate a plurality of locations in a bodily cavity in response to movement of at least part of a transducer-based device (e.g., transducer-based device 200, 300, or 400, in some embodiments) in the bodily cavity. For example, with respect to at least FIG. 2, at least a portion of the transducer-based device (e.g., transducer-based device 200, 300, or 400, according to some embodiments) may be moved or progressed through a sequence of locations in a chamber of the heart or other bodily cavity in the presence of an electric field set (e.g., one or more electric fields generated by the external electrodes (not shown)) or a magnetic field set (e.g., one or more magnetic fields generated by magnetic field generation sources (not shown)). As the portion of the transducer-based device is moved through the sequence of locations, at least some of the catheter's transducers may be configured to generate each location signal set as detected strengths of the respective field(s), which its data processing device system 110, 310 may then be configured to utilize to generate a three-dimensional location of the at least the portion of the catheter (e.g., transducer-based device 200, 300, or 400, according to some embodiments) or its transducers for the respective location in the sequence of locations, according to some embodiments. In this regard, the portion of the transducer-based device may then be moved along the surface of the bodily cavity while providing the location signal sets, which may be used to generate, e.g., according to program instructions associated with block 602b in some embodiments, the envelope 550 in FIG. 5G representative of the surface of the bodily cavity. Intra-cardiac information (discussed in further detail below) may be recorded (e.g., simultaneously) with the location signal sets and correlated to the generated location information in some embodiments. The recorded intra-cardiac information may be represented on the envelope 550, according to some embodiments.

In some embodiments, the graphical representation of the envelope representing a three-dimensional representation of an interior volume of the bodily cavity may be produced with respect to a graphical representation of a pre-existing image or model, such as a CT scan, of the cavity, further assisting the operator to understand the location of the transducer-based device, as well as potential future desired movements of the transducer-based device.

According to some embodiments, methods 600A and 600B may include block 604 associated with computer-executable instructions (e.g., reception instructions provided by a program) configured to cause, via the input-output device system 120, 320, reception of tissue contact information indicating degrees of tissue contact between various transducers of a plurality transducers of a transducer-based device (e.g., 200, 300, 400) and a tissue surface.

Assessing degrees of transducer-to-tissue contact may be motivated for different reasons. In ablation procedures, the degree of transducer-to-tissue contact (e.g., electrode-to-tissue contact) has a bearing on the depth of the lesions formed with fuller or more complete contact typically leading to deeper lesions. Further, in thermal ablation applications, lesser degrees of transducer-to-tissue contact may lead to undesired increased levels of thermal coagulum formation in which at least a portion of the ablation energy is conveyed to blood rather than to tissue. In some electrophysiological activity detection applications, lesser degrees of electrode-to-tissue contact may cause undesired filtering of electric potential information (e.g., intracardiac voltage information). Such filtering may lead to the formation of intracardiac electrograms with reduced sharpness. In some embodiments, each electrode (e.g., 315, 415) is configured to sense or sample an electric potential in the tissue proximate the electrode at a same or different time than delivering energy sufficient for tissue ablation. According to some embodiments, different portions of the transducer-based device (200, 300, 400) are manipulable to in turn manipulate various ones of the plurality of transducers (e.g., transducers 220, 306, 406) into various degrees of contact with a tissue wall within a patient's body.

According to various embodiments, at least some transducers (e.g., at least some of the transducers 220, 306, 406), of the plurality of transducers of the transducer-based device system 200, 300, 400 are arranged in a first spatial distribution (e.g., the spaced apart distribution associated with the deployed configuration of FIGS. 3C, 3D), the distribution positionable in the bodily cavity. The bodily cavity is defined by at least a tissue wall, and, according to various embodiments, each transducer of the at least some transducers, is configured at least to sense a degree of contact between the transducer and the tissue wall. In some embodiments, each particular transducer of the at least some transducers of the transducer-based device 200, 300, 400 may be configured to sense or detect a degree of transducer-to-tissue contact between at least a portion of the particular transducer and the tissue wall. Various methods may be executed to determine the degree of transducer-to-tissue contact including, by way of non-limiting example, techniques including sensing impedance, sensing permittivity, sensing the presence or absence of flow of a fluid (e.g., a bodily fluid), or sensing contact force or pressure. U.S. Pat. No. 8,906,011, issued Dec. 9, 2014, describes example transducer-to-tissue contact sensing techniques. In some embodiments, the tissue-contacting portion of the transducer itself directly senses the degree of tissue contact. In some embodiments, a portion of the transducer other than the tissue-contacting portion of the transducer is configured to sense the degree of contact between the tissue wall and the tissue contacting portion of the transducer. In some embodiments, the tissue-contacting portion of the transducer is provided by an electrode. Referring back to block 602 in FIGS. 6A and 6B, the graphical representation instructions of block 602, block 602a, or block 602b may, in some embodiments, be configured to cause display of information in addition to or other than (a) a graphical representation of at least part of a transducer-based device such as graphical elements associated with transducers, or (b) a representation of at least a portion of a bodily cavity. In some embodiments, program instructions associated with block 602 (or block 602a, block 602b, or both blocks 602a and 602b) in FIG. 6A may include instructions (e.g., input or acquisition instructions included in a program) configured to cause the data processing device system (e.g., data processing device systems 110 or 310) to acquire or receive intra-cardiac information. The graphical representation instructions associated with block 602 (and, in some embodiments, also block 602a, block 602b, or both blocks 602a and 602b) may cause display of various representations of the intra-cardiac information or derivative thereof. Intra-cardiac information can take various forms, including, but not limited to, e.g., electrical information or a derivation thereof (e.g., electrical potential information, such as intra-cardiac electrogram information; electrical impedance information, such as fluidic or non-fluidic cardiac tissue impedance information; electrical conductivity information, such as fluidic or non-fluidic cardiac tissue electrical conductivity), thermal information or a derivation thereof (e.g., temperature information), fluid property information or a derivation thereof (e.g., blood flow information, blood pressure information). In various embodiments, intra-cardiac information may be related to any physiological parameter information related to a heart chamber. In various embodiments, intra-cardiac information may include any information related to, or resulting from an interaction with intra-cardiac tissue. By way of non-limiting example, interaction with intra-cardiac tissue may include an interaction made by way of a diagnostic procedure or treatment procedure.

Intra-cardiac information may be acquired or received by various methods and from various device systems. For example, intra-cardiac information may be received or acquired via data sampling performed by a transducer-based device system (e.g., which may be at least part of the data input-output device system 120, 320) deployed externally from an intra-cardiac chamber or cavity (e.g., outside the chamber or cavity or outside a body including the chamber or cavity). By way of non-limiting example, various “external” transducer-based device systems may include various fluoroscopy device systems, ultra-sound device system, magnetic resonance device systems, computerized tomography device systems, and transthoracic electrocardiographic mapping device systems. In some embodiments, reception or acquisition of the intra-cardiac information may be via data sampling performed by a transducer-based device system (e.g., which may be at least part of the data input-output device system 120, 320) deployed internally to an intra-cardiac chamber or cavity. By way of non-limiting example, various transducer-based device systems that may be internally deployed within an intra-cardiac chamber include by way of non-limiting example transducer-based device systems 200, 300, 400 where data may be sampled by each of one or more transducers of the transducer-based device system, a portion of the transducer-based device system including the one or more transducers positionable in a cardiac chamber during the sampling. Various transducer-based devices may include various intravascularly deployable or percutaneously deployable catheter device systems. Various transducer-based device systems may include detection capabilities, mapping capabilities, diagnostic capabilities, treatment capabilities, or any combination thereof.

In some embodiments, the graphical representation instructions of block 602, block 602a, or block 602b in FIGS. 6A and 6B may, in some embodiments, be configured to cause display of tissue contact information or transducer-to-tissue contact information in addition to or other than (a) a graphical representation of at least part of a transducer-based device such as graphical elements associated with transducers, or (b) a representation of at least a portion of a bodily cavity. In some embodiments, the program instructions associated with block 602 (or block 602a, block 602b, or both blocks 602a and 602b) in FIGS. 6A and 6B may include instructions (e.g., input or acquisition instructions included in a program) configured to cause the data processing device system (e.g., data processing device systems 110 or 310) to cause the display of the tissue contact information based at least on the tissue contact information received per block 604. According to various embodiments, the graphical representation instructions associated with block 602 (and, in some embodiments, also block 602a, block 602b, or both blocks 602a and 602b) may cause display of various representations of the tissue contact information or derivative thereof. In some embodiments, the graphical representation of the transducer-based device and, in some embodiments, its transducers, may have visual characteristics that indicate the various degrees of tissue contact detected by the transducers of the transducer-based device. In some embodiments, the visually indicated various degrees of tissue contact represent degrees of tissue contact presently or currently detected by the transducers. In some embodiments, the graphical representation of the at least the portion of the envelope (e.g., envelope 542 in FIG. 5G, in some embodiments) may include visual characteristics that indicate the various degrees of tissue contact that were detected by the respective transducers when the respective regions of the bodily cavity were mapped. According to some embodiments, an operator is able to concurrently understand the present tissue-contact state exhibited by the transducers by viewing the graphical representation of the transducer-based device and the historical tissue-contact states exhibited by transducers in the past by viewing the graphical representation of the at least the portion of the envelope.

In some embodiments where overlapping graphical representations are displayed, such as a combination of the graphical representation of the transducer-based device, the graphical representation of the envelope representing an interior volume of the bodily cavity, or the graphical representation of the pre-existing image or model of the bodily cavity, blending of colors utilized to represent each of the graphical representations is implemented in a translucent or semi-transparent manner to provide the operator with an efficient understanding of the relative positioning and locational depth (e.g., distance from a viewing perspective or location) of the objects represented by the graphical representations. See, e.g., U.S. Patent Application Publication No. 2021/0353370, published Nov. 18, 2021, for examples pertaining to the generation of and displays including an envelope representing an interior of a bodily cavity.

The displaying of the graphical representation according to the computer-executable instructions associated with block 602 (and, in some embodiments, also block 602a, block 602b, or both blocks 602a and 602b) may, in some embodiments, include causing display of a graphical representation of the tissue contact information or a derivative thereof. Various embodiments may process or analyze (e.g., according to the instructions associated with block 602) the transducer data received by the data processing device system in order to, for example, generate and cause the displayed graphical representation 500 to include (a) intra-cardiac information, (b) tissue contact information, or both (a) and (b). Various embodiments may process or analyze the tissue contact information received by the data processing device system according to block 604 in order to, for example, generate and possibly cause the displayed graphical representation 500 to include a map of at least the tissue contact information. In various embodiments, the tissue contact information is sampled by a transducer-based device system from a plurality of locations in a bodily cavity, which may allow for a mapping of each of a plurality of parts or values of the tissue contact information to a respective one of the plurality of locations in the bodily cavity. In some of these various embodiments, the graphical representation instructions associated with block 602 may be configured to cause an input-output device system (e.g., 120, 320) to display the plurality of parts of the tissue contact information with a first spatial relationship that is consistent with a second spatial relationship between the plurality of locations in the bodily cavity (e.g., a map of the parts of the tissue contact information may be displayed). In some embodiments, the transducer-based device includes a plurality of transducers (e.g., transducer-based device 200, 300) and the sampled tissue contact information may be sampled concurrently from the plurality of locations of the transducers in the bodily cavity.

In FIG. 5H, the graphical representation 500 includes a simple grid of transducer graphical elements 502 represented as squares for ease of illustration. Although the example of FIG. 5H and other examples in various ones of the accompanying figures refer to transducer graphical elements, it should be noted that other types of graphical elements may be utilized in various embodiments to indicate or allow selection of one or more transducers. In this regard, FIG. 5H (as well as each of the figures between FIG. 5H and FIG. 5K, inclusive) may be considered a simplified version of the graphical representations 500 shown in FIGS. 5A-5G, in some embodiments, e.g., for case of illustration. Each square in FIG. 5H is a transducer graphical element 502 in this example (some squares indicated as 502A, 502B, or 502C). According to various embodiments, each of the transducer graphical elements 502 in the graphical representation 500 includes a respective visual characteristic set representative of a degree or level of tissue contact associated with a respective transducer (e.g., 220, 306, or 406 in some embodiments) of the transducer-based device 200, 300, 400, each respective degree or level of tissue contact indicated as per the KEY in FIG. 5H. According to various embodiments, each of the transducer graphical elements 502 in the graphical representation 500 includes a respective visual characteristic set representative of a degree or level of tissue contact having been determined to exist, or having existed between a respective transducer (e.g., 220, 306, or 406 in some embodiments) of the transducer-based device 200, 300, 400 and a tissue surface. According to various embodiments, each of the transducer graphical elements 502 in the graphical representation 500 includes a respective visual characteristic set representative of a degree or level of tissue contact determined at least in part from an analysis of the issue contact information received via block 604.

In FIG. 5H, particular transducer graphical elements 502B (two called out) associated with a first degree or level of tissue contact are indicated with a diagonal hatch pattern. In FIG. 5H, particular transducer graphical elements 502C (two called out) associated with a second degree or level of tissue contact are indicated with a crossed hatch pattern. In FIG. 5H, particular transducer graphical elements 502D (two called out) associated with a third degree or level of tissue contact are indicated with a dotted pattern. In FIG. 5H, particular transducer graphical elements 502A (two called out) associated with no tissue contact are indicated as “blank” with no internal patterning. According to various embodiments, the second degree of tissue contact (e.g., associated with the transducers graphical elements 502C) is greater than the first degree of tissue contact (e.g., associated with the transducers graphical elements 502B). According to various embodiments, each of the first degree of tissue contact (e.g., associated with the transducers graphical elements 502B) and the second degree of tissue contact (e.g., associated with the transducers graphical elements 502C) is greater than the third degree of tissue contact (e.g., associated with the transducer graphical elements 502D).

It is noted that, in some embodiments, indications of tissue contact may be displayed in alternate or additional manners. For example, various regions of space in the graphical representation 500 other than those occupied by the graphical elements 500 may be employed to indicate various degrees or levels of tissue contact. In some embodiments, interpolated values of tissue contact may be indicated based at least on the tissue contact information received as per block 604. In some embodiments, the tissue contact information may be displayed in a spatial map (e.g., FIG. 5H). In some embodiments, the tissue contact information may be displayed in combination with other information (e.g., intra-cardiac information). In some embodiments, the degrees or levels of tissue contact may be indicated in a listing of tissue contact data rather than a spatial map of the tissue contact data.

According to some embodiments, methods 600A and 600B may include block 606 associated with computer-executable instructions (e.g., machine selection instructions (which also may be referred to at least as machine selection instructions) provided by a program) configured to cause, via the input-output device system 120, 320, based at least on an analysis of the tissue contact information (e.g., received as per block 604), a machine selection of a machine-selected group of transducers (e.g., a group of transducers 220, 306, 406). According to various embodiments, the machine selection is an automatic selection made by the data processing device system 110, 310. In some embodiments, the machine or automatic selection of the machine or automatically selected group of transducers is a selection performed by the data processing device system based on an analysis of the tissue contact information without, or without the requirement or necessity of, a user selection of any of the transducers in the group of transducers. According to various embodiments, the machine selection selects the machine-selected group of transducers as having each transducer in the group of transducers being associated with an indication of contact with the tissue surface based at least on the analysis of the tissue contact information. According to various embodiments, the indication of contact with the tissue surface associated with each transducer in the group of transducers indicates contact between the transducer in the group of transducers and the tissue surface at a time in which data pertaining to the tissue contact information was sensed or generated (e.g., by various transducers 220, 306, 406 or other internal or external transducers). According to various embodiments, the indication of contact with the tissue surface associated with each transducer in the group of transducers indicates contact between the transducer in the group of transducers and the tissue surface at a time in which the tissue contact information was received by the data processing device system 110, 310 in accordance with block 604. According to various embodiments, the indication of contact with the tissue surface associated with each transducer in the group of transducers indicates contact between the transducer in the group of transducers and the tissue surface at a time when the analysis of the tissue contact information is conducted. According to various embodiments, the indication of contact with the tissue surface associated with each transducer in the group of transducers indicates existing contact between the transducer in the group of transducers and the tissue surface at a time of the machine selection of the machine-selected group of transducers.

According to various embodiments, the machine selection selects the machine-selected group of transducers as having a degree or level of contact with the tissue surface associated with each transducer in the group of transducers meeting or exceeding a threshold degree of tissue contact as indicated by the analysis of the tissue contact information. According to various embodiments, the threshold degree of tissue contact is a tissue contact degree or level that is likely to be deemed sufficient for a subsequent activation (example, as per block 610a, 610b described further below) of various ones of the transducers (e.g., 220, 306, 406). For example, the threshold degree of tissue contact may mark a degree of tissue contact that is likely to be deemed sufficient to safely and efficaciously form a tissue lesion by tissue ablation performed by the respective transducer, in some embodiments. For another example, the threshold degree of tissue contact may mark a degree of tissue contact that is likely to be deemed sufficient to allow a proper sensing of a tissue characteristic. Accordingly, for example, by machine-selecting transducers that meet or exceed this threshold level of tissue contact, a user can be quickly and efficiently presented with a set of likely viable transducers to perform a successful activation, such as an ablation activation or a sensing activation.

According to various embodiments, the threshold degree of tissue contact is a predetermined threshold degree of tissue contact (e.g., predetermined prior to the reception of the contact information as per block 604 in some embodiments; predetermined prior to the analysis of the analysis of the tissue contact information, in some embodiments). According to various embodiments, the threshold degree of tissue contact is a determined threshold degree of tissue contact. According to various embodiments, the threshold degree of tissue contact is user determined. According to various embodiments, the threshold degree of tissue contact is at least in part machine determined. For example, the data processing device system may, in some embodiments, perform a distribution analysis on the tissue contact information and determine, for instance, the threshold degree of tissue contact to be at a certain location on the distribution.

To elaborate, in some embodiments, the threshold degree of tissue contact is determined (e.g., user determined or machine determined) from the tissue contact information. For instance, a user or the data processing device system may determine the threshold degree of tissue contact from the tissue contact information itself or a derivative of the tissue contact information (e.g., a mapping of the tissue contact information as exemplified in FIG. 5H). A distribution of the degree of tissue contact values may be provided by the tissue contact information or a mapping of the tissue contact information to assess a particular tissue contact threshold likely to result in a successful subsequent activation (example, as per bock 610a, 610b described further below) of various ones of the transducers (e.g., 220, 306, 406).

In some embodiments, the threshold degree of tissue contact indicates at least a partial level or degree of tissue contact (e.g., at least some degree or level of actual contact that may be established between a transducer and a tissue surface, or at least some degree of contact force that may be exerted on a tissue surface by a transducer), and the machine selection selects the machine-selected group of transducers as having a degree or level of contact with the tissue surface associated with each transducer in the group of transducers meeting or exceeding, in various embodiments, the threshold degree of tissue contact as indicated by the analysis of the tissue contact information. In some embodiments, the threshold degree of tissue contact indicates no tissue contact, and the machine selection selects the machine-selected group of transducers as including any transducer of the plurality of transducers having any degree or level of actual contact with the tissue surface (i.e., since the threshold degree of tissue contact indicates no tissue contact in this example). In some embodiments, each transducer of the plurality of transducers 220, 306, 406 meeting or exceeding the threshold degree of tissue contact as indicated by the analysis of the tissue contact information is selected by the machine selection for inclusion in the machine-selected group of transducers. For example, according to some embodiments associated with FIG. 5I, only the transducer graphical elements 502 that correspond to transducers that are associated with degrees of tissue contact that meet or exceed a threshold degree of tissue contact are indicated as machine-selected.

In this regard, FIG. 5I shows, according to some embodiments, the graphical representation 500 of FIG. 5H, modified in accordance with a machine selection made in accordance with block 606. It is noted that, in some embodiments, the machine selection of the machine-selected group of transducers need not be graphically displayed in a map form, such as in FIG. 5I, or may not be displayed at all in some embodiments. In this regard, some embodiments associated with FIG. 5I may be considered illustrative of machine selection aspects of some embodiments of the present invention. The KEY in FIG. 5I includes the degree of contact visual indicator sets employed in FIG. 5H and additionally includes a visual indicator set corresponding to a thick or bolded outline or border of the particular transducer graphical elements 500 that correspond to particular transducers that have been machine selected as per block 606 in FIGS. 6A and 6B. According to various embodiments, each of the transducer graphical elements 502B that correspond to a particular transducer associated with the first degree of tissue contact and each of the transducer graphical elements 502C that correspond to a particular transducer associated with the second degree of tissue contact are indicated (i.e., by a bolded outline) as corresponding to particular transducers that have been machine selected as per block 606. According to various embodiments, each of the transducer graphical elements 502 corresponding to the machine selected transducers include a set of visual characteristics that indicate not only that the transducer graphical element 502 has been machine selected (e.g., via the bolded outline), but also indicate the particular degree of tissue contact associated with the machine selected transducer corresponding to the transducer graphical element 502. For example, in FIG. 5I, the transducer graphical elements 502B include the “machine selected” bolded outline as well as the diagonal hatched pattern indicating the first degree of tissue contact, and the transducer graphical elements 502C include the “machine selected' bolded outline as well as the crossed hatched pattern indicating the second degree of tissue contact. In some embodiments, the transducer graphical elements 502 corresponding to the machine selected transducer may have a set of visual characteristics that indicates the “machine selection” and not the degree of tissue contact.

According to various embodiments, the second degree of tissue contact associated with transducers corresponding to transducer graphical elements 502C is greater than the first degree of tissue contact associated with transducers corresponding to transducer graphical elements 502B, and both the second degree of tissue contact and the first degree of tissue contact are each greater than the third degree of tissue associated with transducers corresponding to transducer graphical elements 502D and greater than the “no-tissue contact” status associated with transducer graphical elements 502A. According to various embodiments, transducer graphical elements 502B and 502C that correspond to the machine-selected group of transducers are associated with, or correspond to, particular transducers of the plurality of transducers that are associated with an indication of contact with the tissue surface based at least on the analysis of the tissue contact information. According to various embodiments associated with FIG. 5I, the threshold degree of tissue contact indicates some degree of actual tissue contact since the transducer graphical elements 502A corresponding to the “no-tissue contact” are not indicated as “machine-selected”. Also indicated in the example of FIG. 5I is that the transducer graphical elements 502D corresponding to the third level of tissue contact also are not indicated as “machine-selected”, but the transducer graphical elements 502B corresponding to the first level of tissue contact are indicated as “machine-selected”. Accordingly, in various embodiments associated with FIG. 5I, it can be seen that the threshold degree of tissue contact is greater than the third degree of tissue contact but is less than or equal to the first degree of tissue contact.

In some embodiments associated with block 606, the machine-selected group of transducers consists of some, but not all, of the transducers of the plurality of transducers. For example, in some embodiments associated with FIG. 5I, each of the transducer graphical elements 502 corresponds to a respective transducer of the plurality of transducers, and the transducer graphical elements 502 that correspond to the machine selected transducers (e.g., the ones shown in bolded outline) are not made up of all of the transducer graphical elements 502 shown in the graphical representation of FIG. 5I.

In some embodiments associated with block 606, the machine selection selects the machine-selected group of transducers with each particular transducer of the group not only meeting or exceeding a threshold degree of tissue contact, but also being selected on the basis of a spatial relationship with other particular transducers in the group. For example, the machine selection may select each transducer in the group of transducers as not only meeting or exceeding a threshold degree of tissue contact but as also being proximate (e.g., adjacent) at least one other particular transducer in the group, according to some embodiments. With reference to FIG. 5I, for example, the machine selection according to block 606 may select all those transducers associated with the bolded outlined transducer graphical elements in FIG. 5I, except those in the subset 510A, because the contiguous block of transducers associated with subset 510A is not adjacent any of the remaining machine-selected transducers according to some embodiments. It is noted that the transducer graphical elements in subset 510A are, according to some embodiments, themselves associated with a group of transducers associated with subset 510A that (i) meet or exceed a threshold degree of tissue contact, and (ii) are each adjacent to another particular transducer associated with the group associated with subset 510 (e.g., contiguously arranged), but is nonetheless not selected as per block 606. This can occur by different mechanisms according to various embodiments. For example, in some embodiments, the group of transducers that is machine-selected as per block 606 is a particular group of multiple candidate groups of transducers (e.g., whose respective transducers meet or exceed a threshold degree of tissue contact and are contiguously arranged) that is adjacent to, at least partially overlies, or at least partially surrounds a particular anatomical region (e.g., a pulmonary vein set). Determination of a positional relationship between each of the candidate groups of transducers and the particular anatomical region may be determined via various techniques including the use of navigation data as described above. In some embodiments, the group of transducers that is machine-selected as per block 606 is a particular group of multiple candidate groups of transducers (e.g., whose respective transducers meet or exceed a threshold degree of tissue contact and are contiguously arranged) that has the greatest number of transducers associated with it. With reference to FIG. 5I, for example, the machine selection according to block 606 may select all those transducers associated with the bolded outlined transducer graphical elements in FIG. 5I, except those in the subset 510A, because the contiguous block of transducers associated with subset 510A are associated with five (5) transducers while the other transducer graphical elements shown as machine selected in FIG. 5I include a group of nineteen (19) contiguous transducers and, therefore, the group of nineteen (19) contiguous transducers would be included in the machine selection instead of the group of five (5) contiguous transducers in this particular example. In some embodiments, the various candidate groups (e.g., whose respective transducers are machine selected as meeting or exceeding a threshold degree of tissue contact and as being contiguously arranged) may be displayed (e.g., sequentially or concurrently) and a user may “toggle” between each of the various candidate groups and choose a desired one (or ones) of the candidate groups as a selected group of transducers. At least some of these various embodiments that allow a user-selection of one or more machine-selected candidate groups may further lead to a more efficient selection of transducers that is better suited for a particular procedure involving the transducers. Additionally, at least some of such embodiments may, in some cases, simplify and reduce the duration of the transducer selection process by reducing the number of selected transducers that must be further added to (e.g., by subsequent user selection of one or more additional transducers) or reduced (e.g., by subsequent user deselection of one or more of the selected transducers) to fine tune the final selected transducer set (for example as described below).

According to some embodiments, at least method 600A may include block 608a associated with computer-executable instructions (e.g., user-deselection instructions provided by a program) configured to cause, via the input-output device system 120, 320 and after the machine selection (as per block 606), reception of user input indicating a user-deselected transducer set. According to various embodiments, each transducer in the user-deselected transducer set is, at the time of deselection, a transducer in the machine-selected group of transducers that is to be deselected. According to various embodiments, the user deselection removes the user-deselected transducer set from the machine-selected group of transducers to form a modified selected transducer set. For example, according to some embodiments, FIG. 5J represents the state of the transducer graphical elements 502 corresponding to the machine-selected group of transducers shown in FIG. 5I after user input indicating a user-deselected transducer set. According to various embodiments associated with FIG. 5J, a first subset 510A of transducers graphical elements 502 corresponding to a first subset of the previously machine-selected transducers that was deselected by the user input is indicated as being deselected. According to various embodiments associated with FIG. 5J, a second subset 510B of transducer graphical elements 502 corresponding to a second subset of the previously machine-selected transducers that was deselected by the user input is indicated as being deselected. The remaining transducer graphical elements 502 corresponding to remaining machine-selected transducers that were not subsequently deselected per block 608a in FIG. 6A are indicated as set 515A in FIG. 5J whose transducer graphical elements correspond to the transducers of the modified selected transducer set in this example. In this regard, according to various embodiments associated with FIG. 5J, the transducer graphical elements 502 in the first subset 510A and the second subset 510B of transducer-graphical elements are indicated as being in a deselected state as the previous bolded outlined border indicating a machine-selected state has been removed (as compared to the state of FIG. 5I). It is noted, however, that in other embodiments, particular transducer graphical elements corresponding to machine-selected transducers, but which are subsequently deselected in response to the user input may be indicated with different visual characteristic sets indicating the deselection. In some embodiments, a visual characteristic set of the remaining transducer graphical elements 502 corresponding to the modified selected transducer set may change in response to the deselection. According to various embodiments, the transducer graphical elements in the first subset 510A and the second subset 510B include, after the deselection, visual characteristic sets that indicate the degree of tissue contact associated with their corresponding transducers.

The user input may indicate the user-deselected transducer set in various ways according to various embodiments. In some embodiments, the user deselection according to block 608a may occur by a user mouse-click or other user interface interaction occurring at the display location of a particular graphical element corresponding to a transducer that is to be deselected, or may occur by a user inputting (e.g., via a keyboard) an identifier associated with the particular graphical element. However, the invention is not limited to any particular manner of deselecting a particular transducer or group of transducers.

According to various embodiments, the plurality of transducers 220, 306, 406 of the transducer-based device 200, 300, 400 are arranged in a spatial distribution, and at least one transducer of the plurality of transducers is located between the user-deselected transducer set and the modified selected transducer set in the spatial distribution. According to some embodiments, the at least one transducer is not included in the user-deselected set. According to some embodiments, the at least one transducer is not included in the modified selected transducer set. For example, in some examples associated with FIG. 5J, the transducer graphical elements 502 are displayed according to a spatial distribution that is consistent with a spatial distribution of the arrangement of the plurality of transducers 220, 306, 406 corresponding to the transducer graphical elements. In FIG. 5J, a transducer graphical element 502A1 is located between the first subset 510A of the transducer graphical elements 502 that corresponds to part of the user-deselected transducer set and the set 515A of transducer graphical elements 502 corresponding to the modified selected transducer set, according to some embodiments. According to various embodiments, transducer graphical element 502A1 corresponds to a transducer of the plurality of transducers that is not included in the user-deselected set and is not included in the modified selected transducer set. According to various embodiments, the plurality of transducers 220, 306, 406 of the transducer-based device 200, 300, 400 are arranged in a spatial distribution, and each particular transducer in the modified selected transducer set is located in the spatial distribution adjacently to another transducer in the modified selected transducer set. For example, in some example embodiments associated with FIG. 5J, the transducer graphical elements 502 are displayed according to a spatial distribution that is consistent with a spatial distribution of the arrangement of the plurality of transducers 220, 306, 406 corresponding to the transducer graphical elements, each transducer graphical element 502 in set 515A is located adjacently to another transducer graphical element in set 515A, each transducer graphical element in the set 515A corresponding to a respective transducer in the modified selected transducer set. According to various embodiments, the transducers in the modified selected transducer set are arranged in a ring-shaped arrangement in the spatial distribution. For example, in FIG. 5J, the spatial arrangement of the transducer graphical elements 502 in set 515A correspond to the spatial arrangement of the transducers 220, 306, 406 in the modified selected transducer set, the spatial arrangement of the transducers 220, 306, 406 in the modified selected transducer set being a ring-shaped arrangement according to various embodiments. The ring-shaped arrangement may be motivated for reasons. For example, ring-shaped arrangements of transducers may be preferred in at least cardiac ablation procedures that involve pulmonary vein isolation, the ring-shaped arrangement of transducers surrounding one or more pulmonary veins to effectively isolate the one or more pulmonary veins after the delivery of tissue ablative energy. In some embodiments, the transducers in the modified selected transducer set surround, in the spatial distribution, a particular transducer set of the plurality of transducers 220, 306, 406. For example, in some embodiments associated with FIG. 5J, the set 515A of transducer graphical elements 502 correspond to the modified selected transducer set which is arranged in a particular arrangement that surrounds a particular transducer set corresponding to transducer graphical elements 502A2. According to various embodiments, each transducer in the particular transducer set is not included in the modified selected transducer set. For example, with reference to FIGS. 5I and 5J, each of transducer graphical elements 502A2 corresponds to a transducer in the particular transducer set that was not machine selected as per block 606 nor was subsequently user deselected as per block 608a. In this regard, according to some embodiments, it may also be considered that each transducer in the particular transducer set is not included in the user-deselected transducer set.

According to various embodiments, the user-deselected transducers set is a user-deselected group of transducers. For example, the first subset 510A of transducer graphical elements 502 and the second subset 510B of transducer graphical elements 502 in FIG. 5J correspond to a user-deselected group of transducers made up of multiple transducers 220, 306, 406. In some embodiments, the number of transducers in the user-deselected transducer set is lower than the number of transducers in the modified selected transducer set. For example, in FIG. 5J, the user-deselected transducer set is exemplified by eight (8) transducer graphical elements 502 among both the first subset 510A and the second subset 510B of transducer graphical elements, as compared with the modified selected transducer set, which is exemplified by sixteen (16) transducer graphical elements 502 in the set 515A of transducer graphical elements. Advantageously, a machine selection of a group of transducers followed by user deselection of a relatively fewer number of the machine selected transducers that may not be required in a specific operation is typically more efficient than if the required transducers were to be manually selected by user, thereby reducing overall procedure time. This is especially so when the desired transducers are affiliated with degrees of tissue contact that are required to exceed a threshold degree of tissue contact. The machine selection of only transducers that are affiliated with degrees of tissue contact that meet or exceed a threshold degree of tissue contact provides for a workflow that is more efficient than if a user was to manually “hunt” for these transducers. Manual user intervention may thus be focused on deselecting (or selecting as discussed below with respect to FIG. 6B) relatively fewer transducers than a conventional workflow. For example, in various embodiments associated with FIG. 5J, the resulting ring-shaped modified selected transducer set may be efficiently selected to perform a pulmonary vein isolation. The machine-based selection of the transducers efficiently ensures that likely sufficient degree of tissue contact is achieved, while the user deselection (or selection discussed below) of a relatively fewer number of transducers to fine-tune the selected arrangement of transducers ensures that only transducers required for an effective pulmonary vein isolation ultimately remain. Accordingly, the risk of deleterious effects, such as stiff left atrial syndrome, may be efficiently reduced along with a reduction of procedure time. It is noted that although the set 515A of transducer graphical elements 502 in FIG. 5J is representative of the modified selected transducer set having a ring-shaped arrangement of selected transducers, according to some embodiments, the transducers in the modified selected transducer set may be arranged in other arrangements, such as at least clustered arrangements in other embodiments. For instance, if the modified selected transducer set was to include only the first subset 510A of transducer graphical elements 502, such may be considered a clustered arrangement, where the transducers are all located close together within a contiguous region. In some embodiments, such a clustered arrangement has no internal gaps like the gap represented by transducer graphical elements 502A2 internal to set 515A in FIG. 5J.

In some embodiments, at least a first transducer in the user-deselected transducer set is not located in the spatial distribution adjacently to any transducer in the modified selected transducer set. For example, in FIG. 5J, a transducer graphical element 502B1 corresponds to a particular transducer included in the user-deselected transducer set, the particular transducer not located adjacently to any transducer in the modified selected transducer set. For instance, transducer graphical element 502A1 is not associated with the user-deselected set and is located between transducer graphical element 502B1 and the set 515A of transducer graphical elements corresponding to the modified selected transducer set, thereby rendering the transducer graphical element 502B1 non-adjacent the set 515A of transducer graphical elements 502, noting that the same applies for the transducers corresponding to the transducer graphical element 502B1 and the set 515A of transducer graphical elements 502, according to various embodiments. For another example, in FIG. 5J, a transducer graphical element 502C1 corresponds to a particular transducer included in the user-deselected transducer set, the particular transducer not located adjacently to any transducer in the modified selected transducer set corresponding to the set 515A set of transducer graphical elements. In some embodiments, at least a second transducer in the user-deselected transducer set is located in the spatial distribution adjacently to at least one transducer in the modified selected transducer set. For example, according to some embodiments associated with FIG. 5J, a second transducer graphical element 502C2 is depicted in the graphical representation 500 adjacently to the set 515A of transducer graphical elements 502, the graphical element 502C2 corresponding to a second transducer 220, 306, 406 in the user-deselected transducer set that is located in the spatial distribution adjacently to at least one transducer in the modified selected transducer set.

In some embodiments, each transducer 220, 306, 406 in the user-deselected transducer set is not located in the spatial distribution adjacently to any transducer in the modified selected transducer set. For example, with reference to FIG. 5J, although the transducer graphical elements in the first subset 510A of transducer graphical elements 502 and the transducer graphical elements in the second subset 510B of transducer graphical elements 502 may combine, according to some embodiments, to form a particular user-deselected transducer set with a particular transducer (e.g., corresponding to transducer graphical element 502C2) located adjacently to the modified selected transducer set (e.g., corresponding to the set 515A of transducer graphical elements 502), user-deselected transducer sets in other embodiments may not include the adjacent particular transducer and may take a form similar to, or the same as, the form corresponding to the first subset 510A of transducer graphical elements 502.

In some embodiments, the user-deselected transducer set is a user-deselected group of transducers. According to various embodiments, the transducers in the modified selected transducer set are arranged in a first particular arrangement in the spatial distribution (for example, an arrangement corresponding to the ring-shaped arrangement of transducer graphical elements in the set 515A of transducer graphical elements 502). In some embodiments, the transducers 220, 306, 406 in the user-deselected group of transducers are arranged in a second particular arrangement in the spatial distribution. In some embodiments, the second particular arrangement is separated from the first particular arrangement in the spatial distribution. For example, with reference to FIG. 5J, although the transducer graphical elements in the first subset 510A of transducer graphical elements 502 and the transducer graphical elements in the second subset 510B of transducer graphical elements 502 may combine, according to some embodiments, to form a particular user-deselected transducer set which includes no separation between a transducer (e.g., corresponding to transducer graphical element 502C2) in the particular user-deselected transducer set and the modified selected transducer set (e.g., corresponding to the set 515A of transducer graphical elements 502), user-deselected transducer sets in other embodiments may be arranged in a separated form from the modified selected transducer set, (e.g., in a manner similar to, or the same as, the form corresponding to the spatial relationship between the first subset 510A of transducer graphical elements 502 and the set 515A of transducer graphical elements 502).

In some embodiments, the second particular arrangement protrudes (e.g., like a peninsula) from the first particular arrangement in the spatial distribution. For example, with reference to FIG. 5J, although the transducer graphical elements in the first subset 510A of transducer graphical elements 502 and the transducer graphical elements in the second subset 510B of transducer graphical elements 502 may combine, according to some embodiments, to form a particular user-deselected transducer set that includes separation between the various transducers corresponding to the first subset 510A of transducer graphical elements 502 and the modified selected transducer set (e.g., corresponding to the set 515A of transducer graphical elements 502), user-deselected transducer sets in other embodiments may be arranged to project from the modified selected transducer set, in some embodiments. For instance, in at least some embodiments where the second particular arrangement corresponds to the user-deselected transducers associated with second subset 510B of transducer graphical elements 502 and where the first particular arrangement corresponds to the modified selected transducer set associated with the set 515A of transducer graphical elements 502 in FIG. 5J, the second particular arrangement protrudes from the first particular arrangement.

According to various embodiments, a user may deselect various ones of the transducers in the machine-selected group of transducers based on factors other than the spatial distribution of various sets of the transducers in the machine-selected group of transducers. For example, the degree of tissue contact associated with a particular transducer in the machine-selected group of transducers may motivate a user to deselect the transducer or allow it to form a part of the modified selected transducer set. For example, in some embodiments, the machine-selected group of transducers includes a first transducer and a second transducer. According to various embodiments, the first transducer may have a first degree of tissue contact as indicated by the analysis of the tissue contact information (e.g., referred to in block 606), and the second transducer may have a second degree of tissue contact as indicated by the analysis of the tissue contact information. According to various embodiments, the second degree of tissue contact is greater than the first degree of tissue contact. In some embodiments, the user-deselected transducer set includes the first transducer. In some embodiments, the user-deselected transducer set excludes the second transducer. For example, working from the example of FIG. 5J, a user may, in some embodiments, deselect each of the transducers associated with the first subset 510A of transducer graphical elements 502 and (in contrast to what is shown in FIG. 5J) not deselect the transducers associated with the second subset 510B of transducer graphical elements 502. In this regard, the transducers associated with the first subset 510A of transducer graphical elements 502 have a lower degree of tissue contact than the transducers associated with the second subset 510B of transducer graphical elements 502. Further, the transducers associated with the first subset 510A of transducer graphical elements 502 are further “isolated” from a desired region of space where the activation is desired to occur (e.g., a region of space associated with set 515A of transducer graphical elements 502), whereas the transducers associated with the second subset 510B of transducer graphical elements 502 are relatively closer (e.g., they project from in this embodiment) to the region space and thus may, in some embodiments, provide some therapeutic benefit if included in the modified selected transducer set. Conversely, in some embodiments, the second transducer may have a second degree of tissue contact as indicated by the analysis of the tissue contact information as being equal to or greater than the first degree of tissue contact, and wherein the user-deselected transducer set may include the second transducer. Accordingly, the choice of whether or not a transducer should be removed from (or added to per at least some embodiments associated with FIG. 6B, discussed below) the machine-selected transducer set may be motivated by various factors that a user may deem to be of relevance during a particular procedure.

Referring to FIG. 6A, some embodiments associated with method 600A may include block 610a associated with computer-executable instructions (e.g., activation instructions provided by a program) configured to cause, via the input-output service system 120, 320, and at least after the machine selection (e.g., as per block 606) and after the reception of the user input indicating the user-deselected transducer set (e.g., as per block 608a), activation of the modified selected transducer set. The activation of the modified selected transducer set may take various forms, according to some embodiments. In some embodiments, the activation of the modified selected transducer set includes concurrent activation of at least two transducers in the modified selected transducer set. In some embodiments, the activation of the modified selected transducer set includes a transmission of energy between at least two transducers in the modified selected transducer set (for example, bipolar activation). The energy transmitted between the at least two transducers in the modified selected transducer set may take various forms. For example, in some embodiments, the energy is sufficient to cause tissue ablation. In some embodiments, the tissue ablation is pulsed field ablation. In some embodiments, the tissue ablation is thermal ablation. In some embodiments, the activation of the modified selected transducer set includes a transmission of energy from each of at least one transducer in the modified selected transducer set, the energy configured to cause tissue ablation. In some embodiments, the tissue ablation is pulsed field ablation. In some embodiments, the tissue ablation is thermal ablation.

In some embodiments, user input may additionally or alternatively indicate transducers that are to be additionally added to the machine-selected transducers for a corresponding transducer activation thereof. For example, in some embodiments associated with at least method 600B a block 608b may be included that is associated with computer-executable instructions (e.g., user selection instructions provided by a program) configured to cause, via the input-output device system 120, 320 and after the machine selection as per block 606, reception of user input indicating a user selection of a user-selected transducer set. According to various embodiments associated with block 608b, each transducer in the user-selected transducer set is not any transducer in the machine-selected group of transducers. According to various embodiments, each transducer in the user-selected transducer set may have less than the threshold degree of tissue contact. For example, FIG. 5K shows the graphical representation 500 of FIG. 5J (i.e., in a state after the user-deselection of at least some of the machine selected transducers) followed by user-selection of four (4) transducers (e.g., transducers 220, 306, 406 in at FIGS. 2, 3C, and 4). In FIG. 5K, four (4) of the five (5) referenced transducer graphical elements 502D correspond to the user-selected transducers, and none of these user-selected transducers formed part of the machine-selected group of transducers shown in FIG. 5I with the respective bolded outlines. According to various embodiments, the user-selected transducer set is a user-selected group of transducers (e.g., a group of four (4) transducers in various embodiments associated with FIG. 5K). In some embodiments, the number of transducers in the user-selected transducer set is lower than the number of transducers in the machine-selected group of transducers (for example, as exemplified in FIG. 5K). According to various embodiments, the machine-selected group of transducers and the user-selected transducer set, in combination, consist of some but not all, of the transducers of the plurality of transducers 220, 306, 406 (for example, as exemplified in FIG. 5K).

According to various embodiments, the user-selected ones of the transducer graphical elements 502D are indicated as user-selected with a visual characteristic set that includes bolded double outlines or borders as indicated by the KEY in FIG. 5K. Other visual characteristic sets may be employed in various embodiments to indicate the user-selection of the user-selected transducer set. For example, in some embodiments, the visual characteristic set indicating a transducer of the user-selected transducer set may be the same as a visual characteristic set of the machine-selected group of transducers (e.g., a bolded single outline or border in FIG. 5K). It is noted that, although the present example of user-selection of additional transducers beyond the machine-selected transducers is based on the state of FIG. 5J which includes a user deselection of transducers, the user selection of the additional transducers need not occur after a user deselection in some embodiments. However, in some embodiments, as with the example of FIG. 5K being subsequent to the state of FIG. 5J, user-deselection of various machine-selected transducers may accompany user-selection of transducers for the user-selected transducer set, and vice versa, according to various embodiments. In summary, according to various embodiments, user deselection of at least one transducer may occur without a user selection of one or more additional transducers, and, according to various embodiments, user deselection of at least one transducer may or may not follow a user selection of one or more additional transducers. And, vice versa, according to various embodiments, user selection of one or more additional transducers may occur without a user deselection of at least one transducer, and, according to various embodiments, user selection of one or more additional transducers may or may not follow a user deselection of at least one transducer.

According to various embodiments, each of the user-selected transducers 220, 306, 406 is associated with a third degree of tissue contact (e.g., as additionally indicated by the corresponding transducer graphical elements 502D via the FIG. 5K KEY). As indicated above in this disclosure, the third-degree of tissue contact is lower than the threshold degree of tissue contact in this example, and also is lower than the first degree of tissue contact and the second degree of tissue contact associated with particular ones of the transducers selected as part of the machine-selected group of transducers. The user selection of each transducer in the user-selected transducer set from transducers having less than the threshold degree of tissue contact may be motivated for different reasons, according to various embodiments. It is noted that in some embodiments, each transducer of the plurality of transducers (or each transducer of the transducer-based device 200, 300, 400 that is configured for operation or activation (e.g., for ablation) within a bodily cavity) meeting or exceeding the threshold degree of tissue contact as indicated by the analysis of the tissue contact information is selected by the machine selection for inclusion in the machine-selected group of transducers, thereby only leaving transducers that are below the threshold degree of tissue contact for user selection for a subsequent activation as per block 610b (described below). If the machine selection has not selected an adequate number of transducers or particular transducers located at a desired location on the basis of the analysis of the tissue contact information, a subsequent user-selection of additional “below threshold degree of contact” transducers may provide some benefits. For example, in some embodiments, the plurality of transducers 220, 306, 406 of the transducer-based device 200, 300, 400 are arranged in a spatial distribution with the machine-selected group of transducers arranged in a ring shaped arrangement in the spatial distribution (for example, as represented by FIG. 5J). According to some embodiments, the transducers in the machine-selected group of transducers and the user-selected transducer set are arranged in a ring-shaped arrangement in the spatial distribution (for example as represented by in FIG. 5K). As compared with FIG. 5J, in FIG. 5K, the user-selection of the additional transducers corresponding to the “user-selected” transducer graphical elements 502D provide more of a complete “ring-shaped” arrangement, according to various embodiments. In ablation procedures such as pulmonary isolation, the more transducers that are activated around a pulmonary vein, the greater the probability that the pulmonary vein may be isolated. In this regard, although not shown in FIG. 5K, yet additional transducers might be selected by a user in appropriate tissue contact and anatomic layout situations to provide a ‘multiple-transducer-wide’ ring around the pulmonary vein to help ensure that the lesion formed by such ring can completely block errant electrical signals passing through the heart tissue. Accordingly, it should be noted that the examples in the figures are merely for purposes of illustration and may not reflect actual ablation or tissue contact patterns.

These machine and user-based selection processing aspects of various embodiments of the present invention allow for an efficient workflow that readily presents a machine selection of transducers that meet or exceed a desired tissue contact threshold and allows revision thereof, if necessary, with user-selected or deselected transducers. It is understood that in various tissue ablation procedures, the user-selected transducers associated with less than desired degrees or levels of tissue contact may not in themselves produce a lesion having a desired quality (e.g., depth, size, etc.), but when combined with the transducers of the machine selection that meet or exceed a desired tissue contact threshold, an overall quality of the combined arrangement of theses transducers may be improved.

It is noted that, in some embodiments associated with FIG. 5K, the transducers in the machine-selected group of transducers and the user-selected transducer set surround a particular transducer set of the plurality of transducers in the spatial distribution (e.g., a set of transducers corresponding to the set of transducer graphical elements 502A2). According to various embodiments, each transducer in the particular transducer set is not included in either the machine-selected group of transducers or the user-selected transducer set. It is noted, however, that spatial arrangements of the transducers in the machine-selected group of transducers and the user-selected transducer set are not limited to a ring-shaped arrangement or encircling arrangements and may take various other forms according to various embodiments. For example, the transducers in the machine-selected group of transducers and the user-selected transducer set may be, in some embodiments, arranged in the spatial distribution in a clustered arrangement.

In some embodiments, at least method 600B may include block 610b associated with computer-executable instructions (e.g., activation instructions provided by a program) configured to cause, via the input-output device system 120, 320, activation of the machine-selected group of transducers and the user-selected transducer set. According to various embodiments, the activation of the machine-selected group of transducers and the user-selected transducer set is initiated in a state in which both the machine-selected group of transducers and the user-selected transducer set are in a selected state (e.g., as in the state of FIG. 5K). In some embodiments, the data processing device system 110, 310 is configured at least by the program at least to (a) record in the memory device system 130, 330 a first indication indicating that the machine-selected group of transducers is in the selected state in response to the machine selection, and (b) record in the memory device system 130, 330 a second indication indicating that the user-selected transducer set is in a selected state in response to the user-selection. In some embodiments, such indications stored in the memory device system 130, 330 may correspond to, facilitate, or cause display of the visual characteristic set of the respective machine-selected (via a bolded single border) or user-selected (via a bolded double border) transducer graphical elements 502, such visual characteristic set utilized to indicate the respective selected statuses of the corresponding transducers, as shown in the example of FIG. 5K. According to various embodiments, the activation of the machine-selected group of transducers and the user-selected transducer set is initiated in a particular state in which the first indication and the second indication concurrently exist as recorded in the memory device system 130, 330, in some embodiments. In some embodiments, this particular state is or includes the state in which both the machine-selected group of transducers and the user-selected transducer set are in the selected state. For instance, in the state of FIG. 5K, the graphical representation of each of the machine-selected transducers as having a bolded outline or border may represent the stored first indication indicating that the machine-selected group of transducers is in the selected state in response to the machine selection of block 606, and the graphical representation of each of the user-selected transducers as having a bolded double outline may represent the stored second indication indicating that the user-selected transducer set is in the selected state in response to the user-selection per block 608b.

In some embodiments, the activation of the machine-selected group of transducers and the user-selected transducer set is caused at least in response to both the machine selection (e.g., per block 606) and the user selection (e.g., per block 608b). For example, in some embodiments, at least the initiation of the activation of both the machine-selected group of transducers and the user-selected transducer set occurs automatically at least in response to both the machine selection (e.g., per at least block 606) and the user selection (e.g., per block 608b). In some embodiments, user confirmation or authorization of the initiation of the activation of both the machine-selected group of transducers and the user-selected transducer set may be required. In this regard, the activation may still be considered to be performed in response to the machine selection (e.g., per block 606) and the user selection (e.g., per block 608b), since such selections at least in part cause energy to flow to the selected transducers once the user confirmation or authorization to activate has been received, according to some embodiments. In some embodiments, the activation of the machine-selected group of transducers and the user-selected transducer set caused at least in response to both the machine selection (e.g., per block 606) and the user selection (e.g., per block 608b) includes concurrent activation of at least a first transducer in the machine-selected group of transducers and at least a second transducer in the user-selected transducer set. In some embodiments, the activation of the machine-selected group of transducers and the user-selected transducer set caused at least in response to both the machine selection (e.g., per at least block 606) and the user selection (e.g., per block 608b) includes an initiation of the activation of at least a first transducer in one of the machine-selected group of transducers and the user-selected transducer set before an initiation of the activation of at least a second transducer in the other of the machine-selected group of transducers and the user-selected transducer set. According to various embodiments, the initiation of the activation of the at least the second transducer in the other of the machine-selected group of transducers and the user-selected transducer set occurs without any user input indicating an activation request of the at least the second transducer being received after the initiation of the activation of the at least the first transducer in one of the machine-selected group of transducers and the user-selected transducer set. For instance, in some embodiments, a user confirmation or authorization to initiate the activation (e.g., per block 610b) of the selected transducers indicated, e.g., in FIG. 5K may result in the initiation of the activation of a sequence of transducers of the selected transducers, such that all selected transducers are activated according to the sequence. In this regard, in some embodiments, once the activation sequence is initiated, no further user input need be required to complete the activation sequence, according to some embodiments.

According to various embodiments, the activation per block 610b of the machine-selected group of transducers and the user-selected transducer set includes concurrent activation of (1) at least one transducer in the machine-selected group of transducers and (2) at least one transducer in the user-selected transducer set. For instance, in the example of FIG. 5K, the activation per block 610b may include concurrent activations of two or more selected transducers in order to, e.g., reduce procedure time for an ablation process or a sensing process by allowing transducers to operate in parallel.

In some embodiments, the activation per block 610b of the machine-selected group of transducers and the user-selected transducer set includes a transmission of energy between (1) at least one transducer in the machine-selected group of transducers and (2) at least one transducer in the user-selected transducer set. For instance, in the example of FIG. 5K, the activation per block 610b may include, e.g., a bipolar ablation activation, where energy is transmitted between the transducer pair associated with transducer graphical elements 502B, 502D, one of which (502B) is machine-selected and one of which (502D) is user-selected, according to some embodiments. The energy transmitted between the at least two selected transducers may take various forms. For example, in some embodiments, the energy is sufficient to cause tissue ablation, as in the aforementioned bipolar ablation example. In some embodiments, the tissue ablation is pulsed field ablation. In some embodiments, the tissue ablation is thermal ablation. While some of the embodiments disclosed above are described with examples of cardiac ablation, the same or similar embodiments may be used for ablating other bodily organs or any lumen or cavity into which the devices of the present invention may be introduced. In some embodiments, the machine-selected group of transducers and the user-selected transducer set (e.g., as shown in the example of FIG. 5K, in some embodiments) form a combined group of transducers, and the activation per block 610b of the machine-selected group of transducers and the user-selected transducer set may include causing a sensing of electrophysiological information by each of at least one transducer in the combined group of transducers, according to some embodiments. In some embodiments, the at least one transducer in the combined group of transducers may include at least a first transducer in the machine-selected group of transducers and at least a second transducer in the user-selected transducer set, e.g., as may be the case if the transducers associated with transducer graphical elements 502B, 502D are activated per block 610b to sense electrophysiological information. As can be seen from the above discussion, the activation associated with block 610b can take various forms of transducer activation according to various embodiments.

While some of the embodiments disclosed above are described with examples of tissue ablation, the same or similar embodiments may be used for mapping various bodily organs, for example, cardiac mapping, gastric mapping, bladder mapping, arterial mapping and mapping of any lumen or cavity into which the devices of the present invention may be introduced. Mapping may include mapping electrophysiological information (for example, in the form of intra-cardiac electrograms).

Subsets or combinations of various embodiments described above can provide further embodiments.

These and other changes can be made to the invention in light of the above-detailed description. In general, in the following claims, the terms used should not be construed to limit the invention to the specific embodiments disclosed in the specification and the claims but should be construed to include other transducer-based device systems including all medical treatment device systems and all medical diagnostic device systems in accordance with the claims. Accordingly, the invention is not limited by the disclosure, but instead its scope is to be determined entirely by the following claims.

What is claimed is:

1. A system comprising:
an input-output device system;
a memory device system storing a program; and
a data processing device system communicatively connected to the input-output device system and the memory device system, the data processing device system configured at least by the program at least to:
cause, via the input-output device system, reception of tissue contact information indicating degrees of tissue contact between various transducers of a plurality of transducers of a transducer-based device and a tissue surface;
cause, based at least on an analysis of the tissue contact information, a machine selection of a machine-selected group of transducers of the plurality of transducers, the machine selection selecting the machine-selected group of transducers as having each transducer in the machine-selected group of transducers being associated with an indication of contact with the tissue surface based at least on the analysis of the tissue contact information and as having each transducer in the machine-selected group of transducers meeting or exceeding a threshold degree of tissue contact as indicated by the analysis of the tissue contact information;
cause, via the input-output device system and after the machine selection, reception of user input indicating a user-deselected transducer set, each transducer in the user-deselected transducer set being a transducer in the machine-selected group of transducers that is to be deselected, the machine-selected group of transducers excluding the user-deselected transducer set forming a modified selected transducer set; and
cause, via the input-output device system and at least after the machine selection and after the reception of the user input indicating the user-deselected transducer set, activation of the modified selected transducer set.

2. The system of claim 1, wherein the machine-selected group of transducers consists of some, but not all, of the transducers of the plurality of transducers.

3. The system of claim 1, wherein each transducer of the plurality of transducers meeting or exceeding the threshold degree of tissue contact as indicated by the analysis of the tissue contact information is selected by the machine selection for inclusion in the machine-selected group of transducers.

4. The system of claim 1, wherein the activation of the modified selected transducer set comprises concurrent activation of at least two transducers in the modified selected transducer set.

5. The system of claim 1, wherein the activation of the modified selected transducer set comprises a transmission of energy between at least two transducers in the modified selected transducer set.

6. The system of claim 5, wherein the energy is configured to cause tissue ablation.

7. The system of claim 6, wherein the tissue ablation is pulsed field ablation.

8. The system of claim 6, wherein the tissue ablation is thermal ablation.

9. The system of claim 1, wherein the activation of the modified selected transducer set comprises a transmission of energy from each of at least one transducer in the modified selected transducer set, the energy configured to cause tissue ablation.

10. The system of claim 9, wherein the tissue ablation is pulsed field ablation.

11. The system of claim 9, wherein the tissue ablation is thermal ablation.

12. The system of claim 1, wherein the activation of the modified selected transducer set comprises causing a sensing of electrophysiological information by each of at least one transducer in the modified selected transducer set.

13. The system of claim 1, wherein the plurality of transducers of the transducer-based device are arranged in a spatial distribution, and wherein at least one transducer of the plurality of transducers is located between the user-deselected transducer set and the modified selected transducer set in the spatial distribution, the at least one transducer not included in the user-deselected transducer set and not included in the modified selected transducer set.

14. The system of claim 1, wherein the plurality of transducers of the transducer-based device are arranged in a spatial distribution, wherein each particular transducer in the modified selected transducer set is located in the spatial distribution adjacently to another transducer in the modified selected transducer set, and wherein at least a first transducer in the user-deselected transducer set is not located in the spatial distribution adjacently to any transducer in the modified selected transducer set.

15. The system of claim 14, wherein at least a second transducer in the user-deselected transducer set is located in the spatial distribution adjacently to at least one transducer in the modified selected transducer set.

16. The system of claim 14, wherein each transducer in the user-deselected transducer set is not located in the spatial distribution adjacently to any transducer in the modified selected transducer set.

17. The system of claim 14, wherein the transducers in the modified selected transducer set are arranged in a ring-shaped arrangement in the spatial distribution.

18. The system of claim 17, wherein the transducers in the modified selected transducer set surround, in the spatial distribution, a particular transducer set of the plurality of transducers, each transducer in the particular transducer set not included in the modified selected transducer set.

19. The system of claim 18, wherein each transducer in the particular transducer set is not included in the user-deselected transducer set.

20. The system of claim 14, wherein the transducers in the modified selected transducer set are arranged in a clustered arrangement in the spatial distribution.

21. The system of claim 1, wherein:
the plurality of transducers of the transducer-based device are arranged in a spatial distribution,
the user-deselected transducer set is a user-deselected group of transducers,
the transducers in the modified selected transducer set are arranged in a first particular arrangement in the spatial distribution, and
the transducers in the user-deselected group of transducers are arranged in a second particular arrangement in the spatial distribution, the second particular arrangement separated from the first particular arrangement in the spatial distribution.

22. The system of claim 1, wherein:
the plurality of transducers of the transducer-based device are arranged in a spatial distribution,
the user-deselected transducer set is a user-deselected group of transducers,
the transducers in the modified selected transducer set are arranged in a first particular arrangement in the spatial distribution, and
the transducers in the user-deselected group of transducers are arranged in a second particular arrangement in the spatial distribution, the second particular arrangement protruding from the first particular arrangement in the spatial distribution.

23. The system of claim 1, wherein the number of transducers in the user-deselected transducer set is lower than the number of transducers in the modified selected transducer set.

24. The system of claim 23, wherein the user-deselected transducer set is a user-deselected group of transducers.

25. The system of claim 1, wherein the machine-selected group of transducers comprises a first transducer and a second transducer, the first transducer having a first degree of tissue contact as indicated by the analysis of the tissue contact information and the second transducer having a second degree of tissue contact as indicated by the analysis of the tissue contact information, the second degree of tissue contact being greater than the first degree of tissue contact, and wherein the user-deselected transducer set includes the first transducer.

26. The system of claim 25, wherein the user-deselected transducer set excludes the second transducer.

27. The system of claim 1, wherein the machine-selected group of transducers comprises a first transducer and a second transducer, the first transducer having a first degree of tissue contact as indicated by the analysis of the tissue contact information and the second transducer having a second degree of tissue contact as indicated by the analysis of the tissue contact information, the second degree of tissue contact being equal to or greater than the first degree of tissue contact, and wherein the user-deselected transducer set includes the second transducer.
